(19) United States
(12) Patent Application Publication
(10) Pub. No.: US 2007/0111930 A1
(43) Pub. Date: May 17, 2007

(54) PROCESS FOR PREPARING VAPREOTIDE

(76) Inventors: Venkata Raghavendra Acharyulu Palle, Hyderabad 500 050, A.P. (IN); Maheedhara Reddy Challa, Hyderabad, A.P. (IN)

Correspondence Address: DR. REDDY'S LABORATORIES, INC., 200 SOMERSET CORPORATE BLVD, SEVENTH FLOOR,, BRIDGEWATER, NJ, 08807-2862, US

(21) Appl. No.: 11/558,550

(22) Filed: Nov. 10, 2006

Related U.S. Application Data
(60) Provisional application No. 60/746,207, filed on May 2, 2006.

(30) Foreign Application Priority Data
Nov. 10, 2005 (IN) 1641/CHE/2005

(51) Int. Cl. A61K 38/12; C07K 7/64

(52) U.S. Cl. 514/9; 530/317

(57) ABSTRACT

A solution phase process for preparing vapreotide, having the formula:

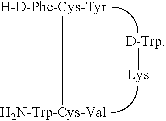

The present invention relates to a process for the preparation of vapreotide or pharmaceutical acceptable salts and intermediates useful therein. More particularly it relates to a process for the preparation of the H-Lys(Boc)-Val-OMe of Formula XII, benzyloxy carbonyl-(D) Trp-Lys(Boc)-Val-OMe of Formula XIV, Boc (D) Phe-Cys (Acm)-Tyr-OH of Formula VIII, and Boc (D) Phe-Cys (Acm)-Tyr-(D) Trp-Lys(Boc)-Val-Cys (Acm)-Trp-NH₂ of Formula XXV, in the liquid phase and conversion to vapreotide.

The amino acid sequence in the octapeptide of the present invention is shown in Formula I (hereinafter the octapeptide is referred to as “vapreotide”).

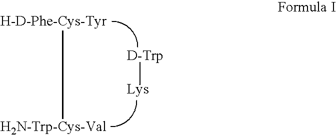

Vapreotide is a somatostatin analogue used for treatment of some tumors, as well as adjuncts to therapy with LH-RH analogues in breast cancer and is available in the form of acetate salt under the brand names Octastatin® and Sanvar®.

In general, the abbreviations used herein for designating the amino acids and the protective groups are based on recommendations of the IUPAC-IUB commission of Biochemical nomenclature. For instance, Abu, Ala, Gly, Cys, Lys, Asn, Asp, Phe, Trp, L-Trp, D-Trp, DI-Trp, D-5-Br-Trp, L-5-F-Trp, Thr, and Ser represent the “residues” of alpha aminobutyric acid, L-alanine, glycine, L-Cysteine, L-lysine, L-asparagine, L-aspartic acid, L-phenylalanine, tryptophan, L-tryptophan, D-tryptophan, DL-tryptophan, D-5-bromotryptophan, L-5-fluorotryptophan, L-threonine, and L-serine respectively.

U.S. Pat. No. 4,725,577 discloses vapreotide, pharmaceutical acceptable salts, compositions containing vapreotide and their use in the treatment of breast cancer. The patent also discloses a process for the preparation of vapreotide in a solid phase such as a resin support, and using fluorenlymethyl chloroformate (hereinafter referred to as “Fmoc”) as the protecting group.

European Patent No. 0 450 480 B1 discloses cyclic somatostatin analogues, and also discloses the octapeptide sequences.

U.S. Patent Application Publication No. 2005/239695 A1 discloses a process for the preparation of disulfide bonds in cyclic peptides comprises first protecting the two free —SH groups with either of trityl or sufonate groups. A subsequent step involves selective deprotection of —S-trityl with anhydrous trifluoroacetic acid and triisopropyl silane in the ratio of 90:10 to 99:1, in the presence of an anhydrous non polar solvent to afford a peptide bond between free —SH and an —SH which is protected with a sulfonate group.

International Application Publication No. 2006/041945 A2 discloses a process for the purification of peptides using a counterion exchange column, and reverse phase HPLC.

The aforementioned processes involve use of solid phase synthetic procedures, which are difficult to scale-up when compared to solution phase procedures.

Hence, there is a need for an industrially suitable process for the preparation of pharmaceutically active compounds such as vapreotide, which will overcome the problems associated with the prior processes.

### SUMMARY OF THE INVENTION

The present invention provides a commercially viable process for the synthesis of vapreotide of Formula I, and its pharmaceutically acceptable salts.

In a first aspect the present invention involves solution phase synthesis of vapreotide using novel protecting groups. Proper selection of the protecting groups is dependent upon the particular succeeding reactions, which must be carried out. The protecting groups used in the present invention are stable both to the reagents and under the conditions employed in the succeeding steps, which are carried out. The protecting groups remain intact during the building of the peptide chain, and can be readily removed upon completion of the synthesis of the desired octapeptide product.

The present invention uses tert-butyldicarbonate (hereinafter referred to as “Boc”) as a protecting group, which is economical, is removable under conditions that do not adversely affect to the rest of the structure being assembled and commercially applicable.

In one aspect, the present invention provides a process for the preparation of vapreotide of Formula I comprising:

a) coupling of Boc (D)-Phe-Cys (PG)-Tyr-OH of Formula VIII with H-(D)Trp-Lys(Boc)-Val-OMe of Formula XV to yield Boc-(D)-Phe-Cys(PG)-Tyr-(D)-Trp-Lys(Boc)-Val-OMe of Formula XXIII;

b) converting Boc-(D)-Phe-Cys(PG)-Tyr-(D)-Trp-Lys(Boc)-Val-OMe of Formula XXIII to Boc-(D)-Phe-Cys(PG)-Tyr-(D)-Trp-Lys(Boc)-Val-OH of Formula XXIV;

c) coupling of Boc-(D)-Phe-Cys(PG)-Tyr-(D)-Trp-Lys(BOC)-Val-OH of Formula XXIV with H-Cys(PG)-Trp-NH₂ of Formula XXII to yield Boc-(D)-Phe-Cys(PG)-Tyr-(D)-Trp-Lys(BOC)-Val-Cys(PG)-Trp-NH₂ of Formula XXV;

d) converting Boc-(D)-Phe-Cys (PG)-Tyr-(D)-Trp-Lys(BOC)-Val-Cys(PG)-Trp-NH₂ of Formula XXV to yield NH₂-(D)Phe-Cys(PG)-Tyr-(D)-Trp-Lys-Val-Cys(PG)-Trp-NH₂ of Formula XXVI, or a salt thereof; and

e) converting NH₂-(D) Phe-Cys (PG)-Tyr-(D)-Trp-Lys-Val-Cys (PG)-Trp-NH₂ of Formula XXVI to vapreotide of Formula I, or a pharmaceutically acceptable salt thereof.

In a second aspect, the present invention provides a process for the preparation of Boc (D)-Phe-Cys(PG)-Tyr-OH of Formula VIII, which is used as an intermediate in the preparation of vapreotide, comprising:

a) coupling Boc-Cys (PG)-OH of Formula II with a salt of H-Tyr-OMe of Formula III to yield Boc-Cys (PG)-Tyr-OMe of Formula IV;

b) converting Boc-Cys (PG)-Tyr-OMe of Formula IV to H-Cys (PG)-Tyr-OMe of Formula V in the presence of an acid, and coupling with Boc-(D)-Phe-OH of Formula VI to yield Boc-(D)-Phe-Cys(PG)-Tyr-OMe of Formula VII; and

c) converting Boc (D)-Phe-Cys (PG)-Tyr-OMe of Formula VII to Boc (D)-Phe-Cys(PG)-Tyr-OH of Formula VIII.

In a third aspect, the present invention provides a process for the preparation of compound H-(D)Trp-Lys(Boc)-Val-OMe of Formula XV, an intermediate used in the preparation of vapreotide comprising:

a) coupling of Val-OMe of Formula IX or a salt thereof with Z-Lys(Boc)-OH of Formula X to yield Z-Lys(Boc)-Val-OMe of Formula XI;

b) converting Z-Lys(Boc)-Val-OMe of Formula XI to H-Lys(Boc)-Val-OMe of Formula XII;

c) coupling of H-Lys(Boc)-Val-OMe of Formula XII with Z-(D)-Trp-OH of Formula XIII to yield Z-(D) Trp-Lys(Boc)-Val-OMe of Formula XIV; and

d) converting Z-(D) Trp-Lys(Boc)-Val-OMe of Formula XIV to H-(D) Trp-Lys(Boc)-Val-OMe of Formula XV.

In a fourth aspect, the present invention provides a process for the preparation of H-Cys (PG)-Trp-NH₂ of Formula XXII, an intermediate used in the preparation of vapreotide comprising:

a) reacting H-Trp-OH of Formula XVI with benzyloxy carbonyl chloride to yield Z-Trp-OH of Formula XVII;

b) reacting Z-Trp-OH of Formula XVII with alkyl chloroformate; then with ammonia to yield Z-Trp-NH₂ of Formula XVIII;

c) converting Z-Trp-NH₂ of Formula XVIII to H-Trp-NH₂ of Formula XIX;

d) coupling of H-Trp-NH₂ of Formula XIX with Boc-Cys (PG)-OH of Formula XX to yield Boc-Cys (PG)-Trp-NH₂ of Formula XXI; and

e) converting Boc-Cys (PG)-Trp-NH₂ of Formula XXI to H-Cys (PG)-Trp-NH₂ of Formula XXII in the presence of an acid.

In a fifth aspect, the present invention provides another process for the preparation of vapreotide of Formula I, and its pharmaceutically acceptable salts, the process comprising:

a) conversion of H (D) Trp-Lys(Boc)-Val-OMe of Formula XXVII in the presence of a base to yield H (D) Trp-Lys(Boc)-Val-OH of Formula XXVIII;

b) conversion of H (D) Trp-Lys(Boc)-Val-OH of Formula XXVIII to Fmoc-(D)-Trp-Lys(Boc)-Val-OH of Formula XXIX using Fmoc.Cl in the presence of a base;

c) coupling of Fmoc-(D)-Trp-Lys(Boc)-Val-OH of Formula XXIX with TFA-Cys (Acm)-Trp-NH₂ to yield Fmoc-(D)-Trp-Lys(Boc)-Val-Cys (Acm)-Trp-NH₂ of Formula XXX;

d) conversion of Fmoc-(D)-Trp-Lys(Boc)-Val-Cys(Acm)-Trp-NH₂ in the presence of a base to yield H-(D)-Trp-Lys(Boc)-Val-Cys(Acm)-Trp-NH₂ of Formula XXXI;

e) coupling of H-(D)-Trp-Lys(Boc)-Val-Cys (Acm)-Trp-NH₂ of Formula XXXI with Boc-(D)-Phe-Cys (Acm)-Tyr-OH to yield Boc-(D)-Phe-Cys (Acm)-Tyr-(D)-Trp-Lys(Boc)-Val-Cys (Acm)-Trp-NH₂ of Formula XXXII;

f) conversion of Boc-(D)-Phe-Cys(Acm)-Tyr-(D)-Trp-Lys(BOC)-Val-Cys(Acm)-Trp-NH₂ of Formula XXXII to NH₂-(D)Phe-Cys(Acm)-Tyr-(D)-Trp-Lys-Val-Cys(Acm)-Trp-NH₂ of Formula XXXIII or salts thereof; and

g) conversion of NH₂-(D) Phe-Cys (Acm)-Tyr-(D)-Trp-Lys-Val-Cys (Acm)-Trp-NH₂ of Formula XXXIII or its salt to vapreotide of Formula I, or a salt thereof.

In a sixth aspect, the present invention provides the compound H-Lys(Boc)-Val-OMe of Formula XII and a process for its preparation.

In a seventh aspect, the present invention provides the compound Z-(D) Trp-Lys(Boc)-Val-OMe of Formula XIV, where Z represents a benzyloxy carbonyl group, and a process for its preparation.

In an eighth aspect, the present invention provides the compound Boc-(D) Phe-Cys (Acm)-Tyr-OH of Formula VIII, and a process for its preparation.

In a ninth aspect, the present invention provides the compound Boc-(D) Phe-Cys(Acm)-Tyr-(D) Trp-Lys(Boc)-Val-Cys(Acm)-Trp-NH₂ of Formula XXV, and a process for its preparation.

### BRIEF DESCRIPTION OF THE DRAWINGS

FIGS. 1A and 1B together are a schematic representation of a process for preparing vapreotide.

FIG. 2 is a schematic representation of a process for preparing Boc (D)-Phe-Cys(PG)-Tyr-OH.

FIG. 3 is a schematic representation of a process for preparing H-(D) Trp-Lys(Boc)-Val-OMe.

FIG. 4 is a schematic representation of a process for preparing H-Cys(PG)-Trp-NH₂.

### DETAILED DESCRIPTION OF THE INVENTION

The present invention provides a commercially viable solution phase process for the synthesis of vapreotide of Formula I, and its pharmaceutically acceptable salts.

In the process descriptions that follow, PG refers to a protecting group for a sulfur atom. Examples of protecting groups that can be used in the present invention include, but are not limited to, N-(hydroxymethyl) acetamide (Acm), triphenyl methanol (Trt), P-methoxy benzyl chloride (Pmb), and Benzyl chloride (Bzl).

Protecting groups which can be used in the protection of nitrogen and oxygen atoms of amino acids include, but are not limited to, benzoyloxycarbonyl groups (Z or cbz), tert-butoxycarbonyl groups (Boc), simple esters such as methyl, and ethyl, benzyl (Bn) esters, acetyl, t-butyl, anisyl, benzyl, trifluoroacetyl, N-hydroxysuccinamide, 4-methyl benzyl, thioanizyl, thiocrysyl, benzyloxymethyl, 4-nitrophenyl, 2-nitrobenzoyl, 2-nitrophenylsulphenyl, 4-toluenesulonyl, diphenylmethyl (Dpm), 2-chlorobenzyloxycarbonyl, triphenyl, methyl and the like.

In one aspect, the present invention provides a process for preparing vapreotide of Formula I and its pharmaceutically acceptable salts. The process comprises:

a) coupling of Boc (D)-Phe-Cys (PG)-Tyr-OH of Formula VIII with H-(D)Trp-Lys(Boc)-Val-OMe of Formula XV 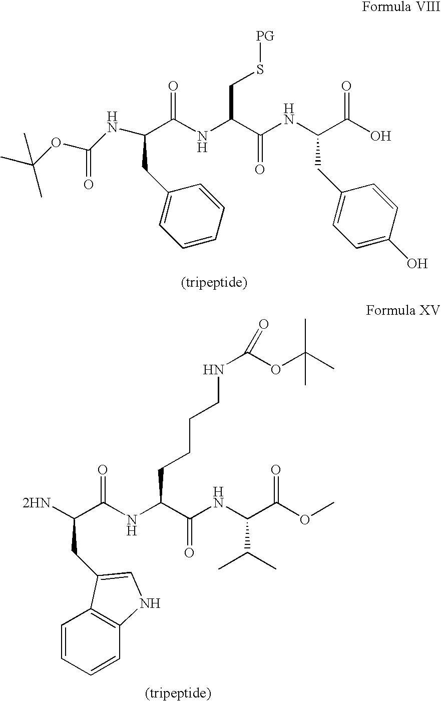
to yield Boc-(D)-Phe-Cys(PG)-Tyr-(D)-Trp-Lys(Boc)-Val-OMe of Formula XXIII;

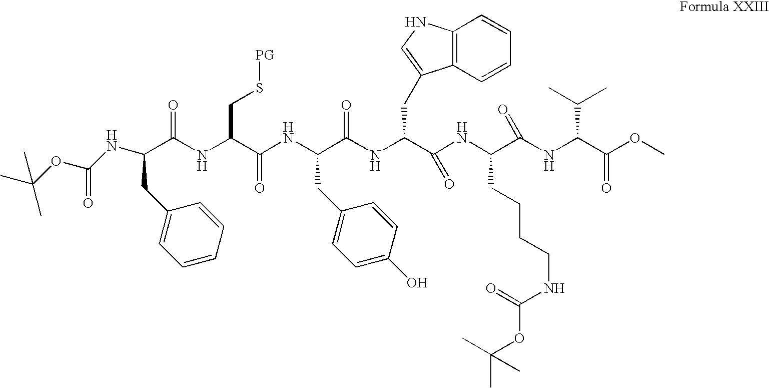

b) converting Boc-(D)-Phe-Cys(PG)-Tyr-(D)-Trp-Lys(Boc)-Val-OMe compound of Formula XXIII to Boc-(D)-Phe-Cys(PG)-Tyr-(D)-Trp-Lys(Boc)-Val-OH of Formula XXIV;

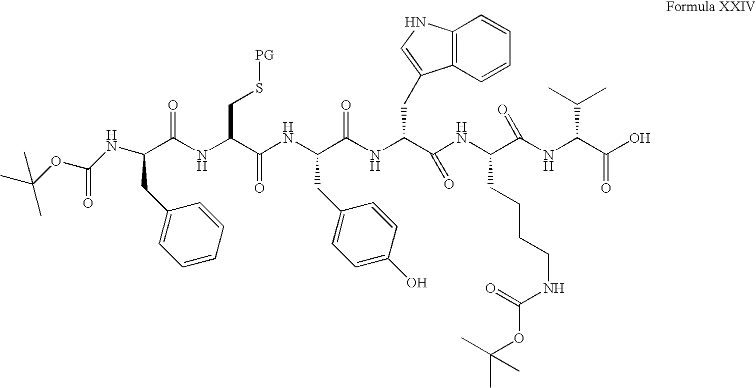

c) coupling of Boc-(D)-Phe-Cys(PG)-Tyr-(D)-Trp-Lys(BOC)-Val-OH of Formula XXIV with H-Cys(PG)-Trp-NH₂ of Formula XXII to yield Boc-(D)-Phe-Cys(PG)-Tyr-(D)-Trp-Lys(BOC)-Val-Cys(PG)-Trp-NH₂ of Formula XXV;

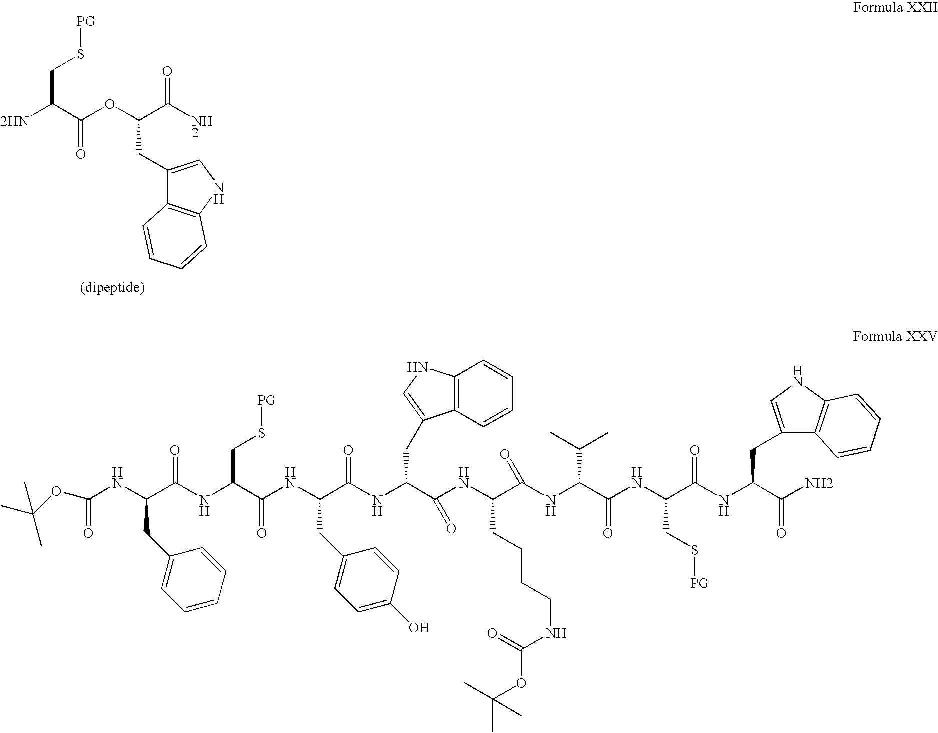

d) converting Boc-(D)-Phe-Cys (PG)-Tyr-(D)-Trp-Lys(BOC)-Val-Cys(PG)-Trp-NH₂ of Formula XXV to yield NH₂-(D)Phe-Cys(PG)-Tyr-(D)-Trp-Lys-Val-Cys(PG)-Trp-NH₂ of Formula XXVI or a salt thereof; and

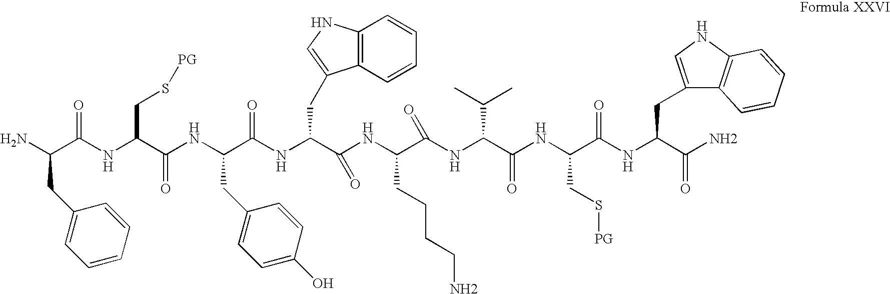

e) converting NH₂-(D) Phe-Cys (PG)-Tyr-(D)-Trp-Lys-Val-Cys (PG)-Trp-NH₂ of Formula XXVI to vapreotide of Formula I, or a pharmaceutically acceptable salt thereof.

Step a) involves coupling of Boc (D)-Phe-Cys (PG)-Tyr-OH of Formula VIII with H-(D)Trp-Lys(Boc)-Val-OMe of Formula XV to yield Boc-(D)-Phe-Cys(PG)-Tyr-(D)-Trp-Lys(Boc)-Val-OMe of Formula XXIII.

Suitable solvents which can be used in the coupling reaction include but are not limited to tetrahydrofuran (THF), 1,4-Dioxane, dimethylformamide (DMF), dimethylsulfoxide (DMSO), ethyl acetate, methyl acetate, acetonitrile, acetone, toluene, chlorinated solvents like dichloromethane (DCM), chloroform, dichloroethane, and the like, and mixture of any two or more of the above solvents.

Suitable temperatures for conducting the reaction may range from about −30° C. to about 100° C., or about −10° C. to about 30° C.

After completion of the reaction, the reaction mixture may be filtered to remove byproducts and the obtained filtrate is concentrated to form a residue.

The formed residue comprising Boc-(D)-Phe-Cys(PG)-Tyr-(D)-Trp-Lys(Boc)-Val-OMe of Formula XXIII may be dissolved in a suitable organic solvent and washed with acid or/and base to remove impurities.

Suitable organic solvents which can be used for the dissolution of the compound having Formula XXIII include but are not limited to ethyl acetate, methyl acetate, n-butyl acetate, dichloromethane, chloroform, and the like.

The organic solution comprising Boc-(D)-Phe-Cys(PG)-Tyr-(D)-Trp-Lys(Boc)-Val-OMe of Formula XXIII may be used directly in the next processing step or it can be concentrated to form a solid.

Step b) involves converting Boc-(D)-Phe-Cys(PG)-Tyr-(D)-Trp-Lys(Boc)-Val-OMe of Formula XXIII to Boc-(D)-Phe-Cys(PG)-Tyr-(D)-Trp-Lys(Boc)-Val-OH of Formula XXIV, by reacting with a suitable base.

Suitable bases which can be used in the conversion of Formula XXIII to Formula XXIV include but are not limited to lithium hydroxide (LiOH), sodium hydroxide, potassium hydroxide, sodium bicarbonate, potassium bicarbonate, sodium carbonate, potassium carbonate and the like. These bases may be used in the form of solids or in the form of aqueous or alcoholic solutions.

If the base is used in the form of an aqueous or alcoholic solution, then suitably from about 0.5 N to about 2 N solutions may be used

Suitable solvents that can be used in the step of b) include but are not limited to: dichloromethane, chloroform and the like; ethers such as THF, diethyl ether, diisopropyl ether, 1,4-Dioxane and the like; esters such as ethyl acetate, methyl acetate, and the like; and aromatic solvents such as benzene, toluene, and the like.

After reaction completion, the reaction mixture is diluted with water and washed with a suitable water immiscible solvent to remove impurities.

Suitable solvents which can be used in the washing include but are not limited to ethers such as diethyl ether, diisopropyl ether, and the like; and hydrocarbons such as n-hexane, cyclohexane, heptane and the like.

The aqueous layer comprising the compound Boc-(D)-Phe-Cys(PG)-Tyr-(D)-Trp-Lys(BOC)-Val-OH of Formula XXIV is treated with a suitable acid and extracted into a suitable organic solvent.

Suitable acids that can be used include but are not limited to inorganic acids such as potassium bisulfate, hydrochloric acid, hydrobromic acid and the like. Suitably, the reaction mixture pH may be adjusted to about 1 to about 6.

Suitable organic solvents which can be used for extraction include but are not limited to esters such as ethyl acetate, methyl acetate, n-butyl acetate, ethylacetoacetate, and the like; chlorinated solvents such as dichloromethane, dichloroethane, chloroform, and the like; ethers such as diethyl ether, diisopropyl ether, and the like; and hydrocarbons such as n-hexane, cyclohexane, heptane and the like.

The organic solution comprising Boc-(D)-Phe-Cys(PG)-Tyr-(D)-Trp-Lys(BOC)-Val-OH of Formula XXIV may be used directly in the next processing step or it can be concentrated to form a residue.

The compound of Boc-(D)-Phe-Cys(PG)-Tyr-(D)-Trp-Lys(Boc)-Val-OH of Formula XXIV may be purified by using crystallization or reslury techniques if required.

Suitable solvents that can be used in the purification technique include but are not limited to: esters such as ethyl acetate, methyl acetate, isobutyl acetate and the like; ethers such as diethyl ether, dimethyl ether, diisopropyl ether, tetrahydrofuran, 1,4-dioxane, and the like; and chlorinated solvents such as dichloromethane, chloroform and the like.

Step c) involves coupling of Boc-(D)-Phe-Cys(PG)-Tyr-(D)-Trp-Lys(BOC)-Val-OH of Formula XXIV with H-Cys(PG)-Trp-NH₂ of Formula XXII to yield Boc-(D)-Phe-Cys(PG)-Tyr-(D)-Trp-Lys(BOC)-Val-Cys(PG)-Trp-NH₂ of Formula XXV, in the presence of a suitable reagent.

Suitable temperatures for conducting the reaction may range from about −30° C. to about 100° C., or about −10° C. to about 30° C.

The compound H-Cys(PG)-Trp-NH₂ of Formula XXII, which is used in the preparation of Boc-(D)-Phe-Cys(PG)-Tyr-(D)-Trp-Lys(Boc)-Val-Cys(PG)-Trp-NH₂ of Formula XXV, is prepared by a process comprising the reaction of Boc-Cys (PG)-Trp-NH₂ of Formula XXI in the presence of an acid.

Suitable acids which can be used in the preparation of Formula XXII include but are not limited to acetic acid, trifluoroacetic acid (TFA), hydrochloric acid, sulphuric acid and the like.

After completion of the reaction, H-Cys(PG)-Trp-NH₂ of Formula XXII is isolated in the form of the corresponding acid addition salt. The acid addition salt of H-Cys(PG)-Trp-NH₂ of Formula XXII is converted into the free base by treating with a suitable base such as N-methyl morpholine, ammonia and the like.

The quantity of base that can be used for conversion of salt to free base is about 2 to about 5 molar equivalents per equivalent of acid addition salt of H-Cys(PG)-Trp-NH₂ of Formula XXII.

The resulting compound H-Cys(PG)-Trp-NH₂ of Formula XXII is reacted with Boc-(D)-Phe-Cys(PG)-Tyr-(D)-Trp-Lys(BOC)-Val-OH of Formula XXIV in the presence of a suitable reagent.

Suitable temperatures for conducting the reaction range from about −30° C. to about 100° C., or about −10° C. to about 30° C.

Step d) involves converting Boc-(D)-Phe-Cys (PG)-Tyr-(D)-Trp-Lys(BOC)-Val-Cys(PG)-Trp-NH₂ of Formula XXV to NH₂-(D)Phe-Cys(PG)-Tyr-(D)-Trp-Lys-Val-Cys(PG)-Trp-NH₂ of Formula XXVI, or a salt thereof, by reacting with an acid.

Suitable acids that can be used in the formation of Formula XXVI include but are not limited to trifluoroacetic acid, hydrochloric acid, benzene sulfonic acid and the like, in the presence of thioanisole.

Suitable solvents that can be used in step d) include but are not limited to: dichloromethane, chloroform and the like; ethers such as THF, diethyl ether, diisopropyl ether, 1,4-Dioxane and the like; esters such as ethyl acetate, methyl acetate, and the like; and aromatic solvents such as benzene, toluene, and the like.

Suitable temperatures for conducting the reaction range from about −30° C. to about 100° C., or about −10° C. to about 30° C.

Suitable salts which can be formed in the above reaction include but are not limited to the hydrochloride salt, trifluoroacetate salt, mesylate salt, besylate salt, and the like.

The compound NH₂-(D)Phe-Cys(PG)-Tyr-(D)-Trp-Lys-Val-Cys(PG)-Trp-NH₂ of Formula XXVI or its salt may be purified, using recrystallization or slurrying techniques.

Suitable solvents that can be used in the purification include but are not limited to alcoholic solvents such as methanol, ethanol, isopropyl alcohol and the like or mixtures of solvents.

Step e) involves converting NH₂-(D) Phe-Cys (PG)-Tyr-(D)-Trp-Lys-Val-Cys (PG)-Trp-NH₂ of Formula XXVI to vapreotide of Formula I or a pharmaceutically acceptable salt thereof, in the presence of an acid.

Suitable acids that can be used in the formation of vapreotide of Formula I or salts thereof include but are not limited to acetic acid, trifluoroacetic acid, hydrochloric acid, benzene sulfonic acid and the like; in the presence of iodine.

The concentration of the acid that can be used in step e) is in the range of about 0.01 to about 1% in water.

Suitable solvents that can be used in step e) include but are not limited to: water; alcoholic solvents such as methanol, ethanol, isopropyl alcohol, and the like; nitriles such as acetonitrile and the like; chlorinated solvents such as dichloromethane, chloroform and the like; ethers such as THF, diethyl ether, diisopropyl ether, 1,4-dioxane and the like; esters such as ethyl acetate, methyl acetate, and the like; and aromatic solvents such as benzene, toluene, and the like.

After completion of the reaction, reaction mass is quenched with sodium thiosulphate solution and the compound is purified using suitable purification techniques.

Suitable purification techniques that can be used in the above step include but are not limited to preparative high performance liquid chromatography, flash column chromatography, precipitation using antisolvnet, and the like.

The purification of vapreotide can be achieved by recrystallization, reslury or a combination thereof, or preparative high performance liquid chromatography, and flash column chromatography, typically followed by drying of the resulting wet compound.

Suitable solvents which can be used in the purification include but are not limited to: water; alcoholic solvents such as methanol, ethanol, isopropyl alcohol, n-butanol and the like; nitriles such as acetonitrile, and the like; chlorinated solvents such as dichloromethane, chloroform and the like; and esters such as ethyl acetate, methyl acetate, butyl acetate and the like.

The solid can be isolated by conventional techniques such as filtering, decanting, centrifuging and the like, including filtering using gravity or a vacuum, optionally under an inert atmosphere using gases such as for example nitrogen and the like.

The wet cake obtained may optionally be further dried. Drying can be suitably carried out in a tray dryer, vacuum oven, air oven, fluidized bed drier, spin flash dryer, flash dryer, freeze drier and the like. The drying can be carried out at temperatures appropriate for the solvent being used, such as but not limited to temperatures of about 35° C. to about 70° C. The drying can be carried out for any desired time periods until the desired product purity is obtained; frequently times from about 1 to 20 hours, or longer, are used.

Suitable salts which can be formed in the above reaction include but are not limited to the hydrochloride salt, trifluoroacetate salt, mesylate salt, besylate salt and the like.

Examples of coupling reagents which can be used in the steps a) and c) include but are not limited to carbodiimide compounds like 1-[3-(dimethylamino)propyl]-3-ethylcarbodiimide hydrochloride (EDC), N-cyclohexyl-N′-isopropylcarbodiimide (CIC), N,N′-diisopropylcarbodiimide (DIC), and the like.

Suitable reagents which can be used in the steps a) and c) to avoid racemization include but are not limited to 1-hydroxybenzotriazole hydrate, ethyl-1-hydroxy-1H-1,2,3-triazole-4-carboxylate (HOCt), and N-hydroxysuccinimide (HOSu); imidazloium reagents like 1,1′-carbonyldiimidazole (CDI), 1,1′-carbonylbis(3-methyl-imidazolium)triflate (CBMIT); 2-(benzotriazol-1-yl)oxy-1,3-dimethyl-imidazolidinium hexafluorophosphate (BOI), CMBI; mixed anhydride coupling reagents like Iso-butyl chloroformate (IBCF), ethyl chloroformate (ECF), Iso-propyl chloroformate (IPCF), PhO2CCl; uronium reagents like O-(benzotriazol-1-yl)-1,1,3,3-tetramethyluronium hexafluorophosphate (HBTU), O-benzotriazol-1-yl-1,1,3,3-tetramethyluronium tetrafluoroborate (TBTU), 2-succinimido-1,1,3,3-tetramethyluronium tetrafluoroborate (TSTU), 2-(5-norbornene-2,3-dicarboximido)-1,1,3,3-tetramethyluronium tetrafluoroborate (TNTU); phosphonium reagents like BOP, Bromotri (pyrrolidino) phosphonium hexafluorophosphate (PyBrop), Benzotriazol-1-yloxytri(pyrrolidino)phosphonium hexafluorophosphate (PyBOP), and chlorotri(pyrrolidino)phosphonium hexafluorophosphate (PyClOP) and the like.

Suitable solvents that can be used in the in the steps a) and c) include but are not limited to ethers such diethyl ether, diisopropylether, tetrahydrofuran, 1,4-dioxane, and the like; esters such as ethyl acetate, methyl acetate, n-butyl acetate, and the like; chlorinated solvents such as dichloromethane, chloroform and the like; hydrocarbons such as hexane, heptane and the like; other solvents like dimethylformamide, dimethylsulphoxide, acetonitrile, acetone, and toluene; and combinations thereof. Bases, which can be used in the neutralization of peptides, include but are not limited to N-methylmorpholine, ammonia and the like.

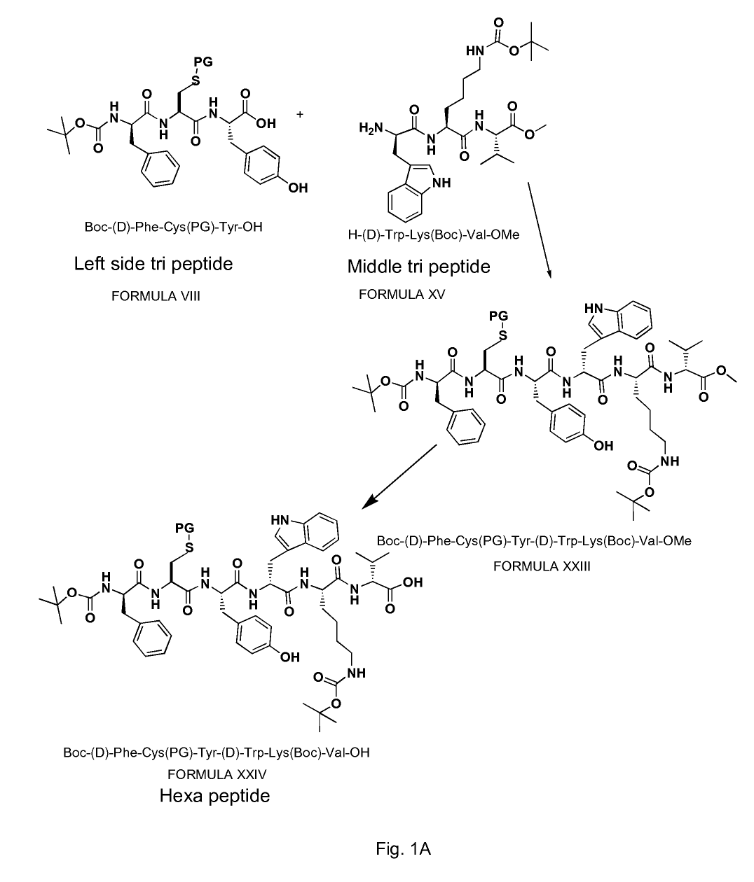

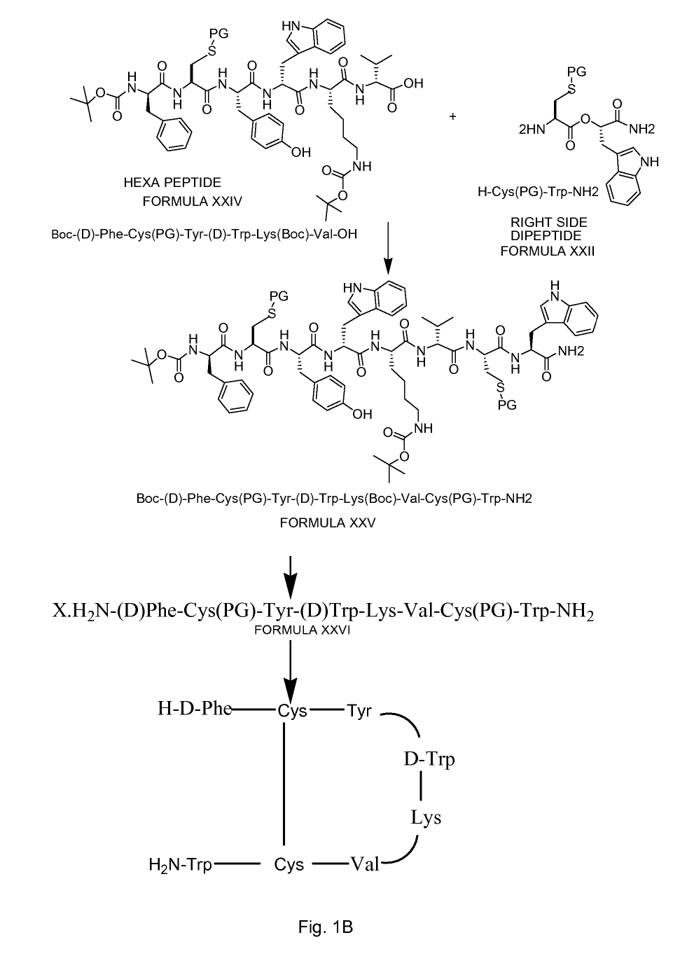

The above-discussed process is represented schematically in FIGS. 1A and 1B.

In a second aspect, the present invention provides a process for the preparation of Boc (D)-Phe-Cys(PG)-Tyr-OH of Formula VIII, which is used as intermediate in the preparation of vapreotide, comprising:

a) coupling Boc-Cys (PG)-OH of Formula II with a salt of H-Tyr-OMe of Formula III to yield Boc-Cys (PG)-Tyr-OMe of Formula IV;

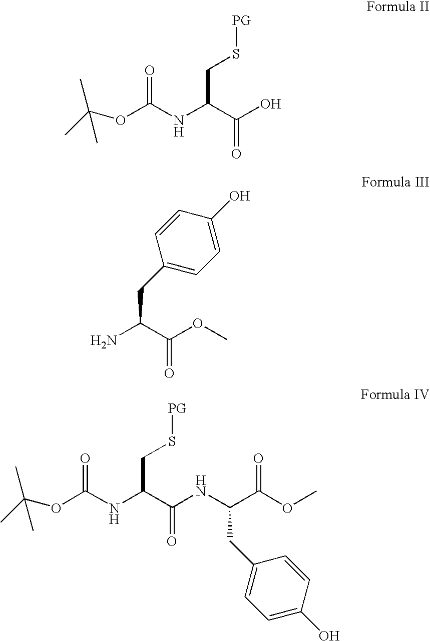

b) converting Boc-Cys (PG)-Tyr-OMe of Formula IV to H-Cys (PG)-Tyr-OMe of Formula V in the presence of an acid, and coupling with Boc-(D)-Phe-OH of Formula VI to yield Boc-(D)-Phe-Cys(PG)-Tyr-OMe of Formula VII; and

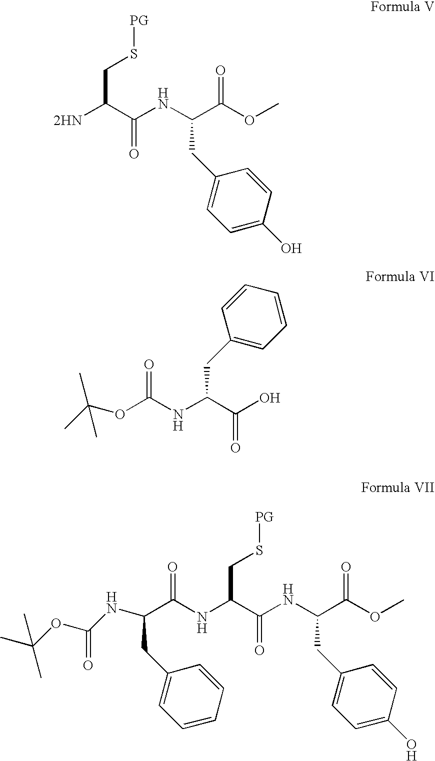

c) converting Boc (D)-Phe-Cys (PG)-Tyr-OMe of Formula VII to Boc (D)-Phe-Cys(PG)-Tyr-OH of Formula VIII in the presence of an acid.

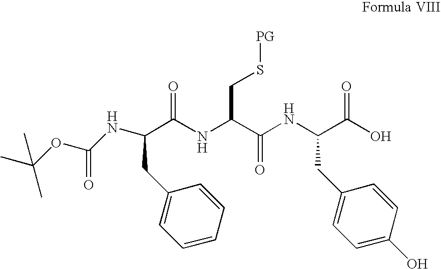

Step a) involves coupling Boc-Cys (PG)-OH of Formula II with a salt of H-Tyr-OMe of Formula III to yield Boc-Cys (PG)-Tyr-OMe of Formula IV.

If H-Tyr-OMe of Formula III is used in the form of a salt, it is converted into the free base using a suitable base. Suitable bases, which can be used in the preparation of the free base, include but are not limited to N-methyl morpholine, ammonia and the like.

The quantity of base that can be used in the neutralization is about 2 to about 5 molar equivalents, per equivalent of salt of H-Tyr-OMe of Formula III.

Suitable temperatures for conducting the reaction may range from about −30° C. to about 100° C., or about 0° C. to about 30° C.

After completion of the reaction, the reaction mixture is filtered to remove byproducts and washed with a suitable solvent.

Suitable solvents, which can be used in the washing to remove impurities, include but are not limited to ethers such as diethyl ether, diisopropyl ether, 1,4-dioxane, THF, and the like;

The formed reaction mixture may be washed with an acid to extract the compound into an aqueous solvent and washed with a base to extract the compound into an organic layer.

Suitable acids that can be used in the above step include but are not limited to hydrochloric acid, hydrobromic acid, sulfuric acid, and the like.

The concentration of acid that can be used in the above reaction is to about 0.1 to about 2N in water.

Suitable bases that can be used in the above step for washing include but are not limited to sodium carbonate, potassium carbonate, sodium bicarbonate, potassium bicarbonate and the like.

These bases can be used in form of solid or in the form of aqueous or in the form of alcoholic solution.

The concentration of base that can be used in the above reaction is to about 5% to about 20% in water.

The organic solution comprising Boc-Cys (PG)-Tyr-OMe of Formula IV may be used directly in the next processing step or it can be concentrated to form a residue.

Step b) involves converting Boc-Cys (PG)-Tyr-OMe of Formula IV to H-Cys (PG)-Tyr-OMe of Formula V in the presence of an acid, and coupling with Boc-(D)-Phe-OH of Formula VI to yield Boc-(D)-Phe-Cys(PG)-Tyr-OMe of Formula VII.

Suitable acids which can be used in the conversion of Formula IV to Formula V include but are not limited to acetic acid, triflouroacetic acid, hydrochloric acid and the like.

Suitable solvents that can be used in the step of b) include but are not limited to: dichloromethane, chloroform and the like; ethers such as THF, diethyl ether, diisopropyl ether, 1,4-dioxane and the like; esters such as ethyl acetate, methyl acetate, and the like; and aromatic solvents such as benzene, toluene, and the like.

After completion of the reaction, the reaction mixture comprising H-Cys (PG)-Tyr-OMe of Formula V may be concentrated to get residue and dissolved in a suitable solvent, then adjusting pH to about 7 to about 9 with a base.

Suitable bases that can be used in the pH adjustment include but are not limited to N-methylmorpholine, ammonia, sodium bicarbonate, potassium bicarbonate, sodium hydroxide, potassium hydroxide, sodium carbonate, potassium carbonate and the like.

Suitable solvents which can be used in the dissolution of H-Cys (PG)-Tyr-OMe of Formula V include but are not limited to ethers such as diethyl ether, dimethyl ether, diisopropyl ether, THF, 1,4-dioxane and the like.

H-Cys (PG)-Tyr-OMe of Formula V is coupled with Boc-(D)-Phe-OH of Formula VI to yield Boc-(D)-Phe-Cys(PG)-Tyr-OMe of Formula VII in the presence of an alkyl chloroformate and ammonia.

Alkyl chloroformates which can be used in the coupling reaction include but are not limited to methylchloroformate, ethylchloroformate, isobutylchloroformate, and the like.

Suitable temperatures for conducting the reaction range from about −30° C. to about 100° C., or about 0° C. to about 30° C.

After completion of the reaction, water and organic solvent are charged to the reaction mixture and the compound of Formula VII extracted into the organic solvent.

Suitable solvents that can be used in the extraction include but are not limited to tetrahydrofuran (THF), 1,4-Dioxane, dimethyl formamide (DMF), dimethylsulfoxide (DMSO), ethyl acetate, methyl acetate, acetonitrile, acetone, toluene, chlorinated solvents like dichloromethane (DCM), chloroform, dichloroethane, and the like, and mixtures of the above solvents.

The organic solution comprising Boc (D)-Phe-Cys (PG)-Tyr-OMe of Formula VII may be used directly in the next processing step or it can be washed with hydrochloric acid, then with sodium bicarbonate solution, and concentrated completely to form a solid.

Boc (D)-Phe-Cys (PG)-Tyr-OMe of Formula VII may be purified by recrystallization or by a slurrying technique.

Suitable solvents which can be used in the purification include but are not limited to: esters such as ethyl acetate, ethyl acetoacetate, n-butyl acetate and the like; ethers such as dimethyl ether, diethyl ether, diisopropyl ether, THF, and 1,4-dioxane; petroleum ether; and the like.

Step c) involves converting Boc (D)-Phe-Cys (PG)-Tyr-OMe of Formula VII to Boc (D)-Phe-Cys(PG)-Tyr-OH of Formula VIII in the presence of a base.

Suitable bases that can be used in step c) include but are not limited to sodium hydroxide, potassium hydroxide, sodium carbonate, potassium carbonate, sodium bicarbonate, potassium bicarbonate and the like. The suitable bases can be used in form of solids or in the form of aqueous or alcoholic solutions.

The concentration of bases, which can be used in the above reaction, is to about 0.5 to about 1N.

Suitable solvents that can be used in step c) include but are not limited to: dichloromethane, chloroform and the like; ethers such as THF, diethyl ether, diisopropyl ether, 1,4-dioxane and the like; esters such as ethyl acetate, methyl acetate, and the like; and aromatic solvents such as benzene, toluene, and the like.

Suitable temperatures for conducting the reaction range from about −30° C. to about 60° C., or about 0° C. to about 15° C.

After completion of the reaction, the reaction mixture is treated with a acid to adjust the reaction mixture pH to about 2 to about 5.

Suitable acids that can be used in the above pH adjustment include but are not limited to potassium hydrogen sulphate, and the like.

The obtained organic solution can be extracted into a suitable organic solvent and concentrated to form a solid. The formed solid optionally, purified, using recrystallization and reslury techniques.

Suitable solvents that can be used in the purification include but are not limited to ethers such as tetrahydrofuran (THF), 1,4-dioxane, diethyl ether, diisopropyl ether, petroleum ether, and the like; and esters such as ethyl acetate, methyl acetate, n-butyl acetate and the like.

The coupling reagents which can be used in the steps a) and b) include but are not limited to carbodiimide reagents like 1-[3-(dimethylamino)propyl]-3-ethylcarbodiimide hydrochloride (EDC), N-cyclohexyl-N′-isopropylcarbodiimide (CIC), N,N′-diisopropylcarbodiimide (DIC), and the like.

Suitable reagents which can be used in the steps a) and b) to avoid racemization include but are not limited to 1-hydroxybenzotriazole hydrate, ethyl-1-hydroxy-1H-1,2,3-triazole-4-carboxylate (HOCt), and N-hydroxysuccinimide (HOSu); imidazolium reagents like 1,1′-carbonyldiimidazole (CDI), 1,1′-carbonylbis(3-methyl-imidazolium)triflate (CBMIT); 2-(benzotriazol-1-yl)oxy-1,3-dimethyl-imidazolidinium hexafluorophosphate (BOI), CMBI; mixed anhydride coupling reagents like Iso-butyl chloroformate (IBCF), ethyl chloroformate (ECF), iso-propyl chloroformate (IPCF), PhO2CCl; uronium reagents like O-(benzotriazol-1-yl)-1,1,3,3-tetramethyluronium hexafluorophosphate (HBTU), O-benzotriazol-1-yl-1,1,3,3-tetramethyluronium tetrafluoroborate (TBTU), 2-succinimido-1,1,3,3-tetramethyluronium tetrafluoroborate (TSTU), 2-(5-norbornene-2,3-dicarboximido)-1,1,3,3-tetramethyluronium tetrafluoroborate (TNTU); phosphonium reagents like BOP, Bromotri (pyrrolidino) phosphonium hexafluorophosphate (PyBrop), Benzotriazol-1-yloxytri(pyrrolidino)phosphonium hexafluorophosphate (PyBOP), and Chlorotri(pyrrolidino)phosphonium hexafluorophosphate (PyClOP) and the like.

Suitable solvents that can be used in steps a) and b) include but are not limited to: ethers such diethyl ether, diisopropyl ether, tetrahydrofuran, 1,4-dioxane, and the like; esters such as ethyl acetate, methyl acetate, n-butyl acetate, and the like; chlorinated solvents such as dichloromethane, chloroform and the like; hydrocarbons such as hexane, heptane and the like; other solvents like dimethylformamide, dimethylsulphoxide, acetonitrile, acetone, and toluene; and combinations thereof. Bases, which can be used in the neutralization of peptides, include but are not limited to N-methylmorpholine, ammonia and the like.

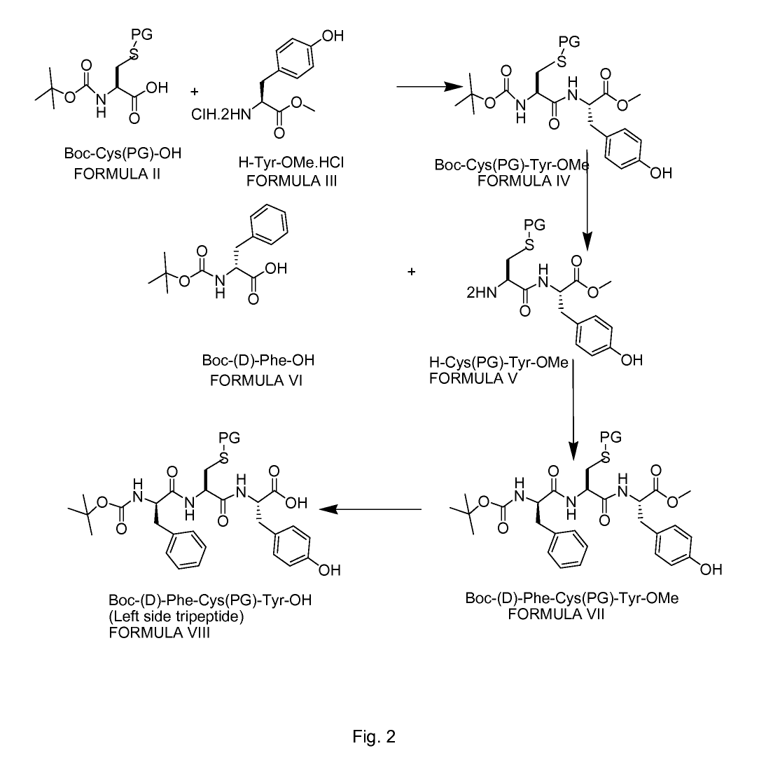

The above mentioned process is represented schematically in FIG. 2.

In a third aspect, the present invention provides a process for the preparation of compound H-(D)Trp-Lys(Boc)-Val-OMe of Formula XV, an intermediate used in the preparation of vapreotide, comprising:

a) coupling Val-OMe of Formula IX or a salt thereof with Z-Lys(Boc)-OH of Formula X to yield Z-Lys(Boc)-Val-OMe of Formula XI;

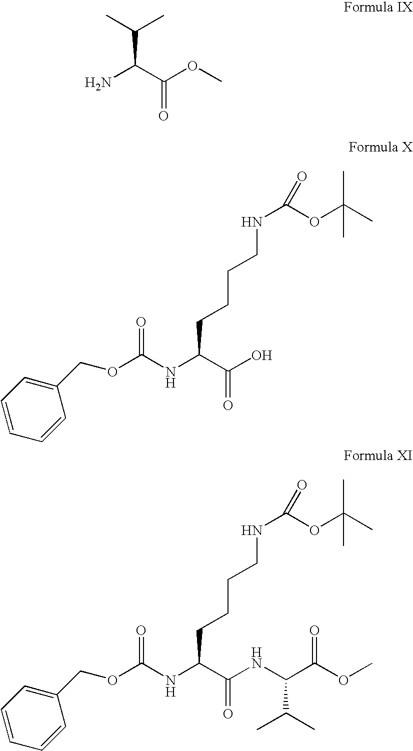

b) converting Z-Lys(Boc)-Val-OMe of Formula XI to H-Lys(Boc)-Val-OMe of Formula XII;

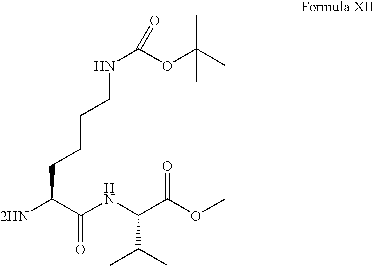

c) coupling of H-Lys(Boc)-Val-OMe of Formula XII with Z-(D)-Trp-OH of Formula XIII to yield Z-(D) Trp-Lys(Boc)-Val-OMe of Formula XIV; and

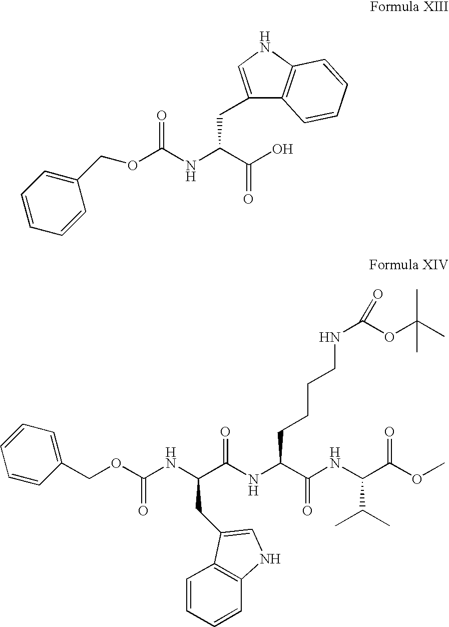

d) converting Z-(D) Trp-Lys(Boc)-Val-OMe of Formula XIV to H-(D) Trp-Lys(Boc)-Val-OMe of Formula XV.

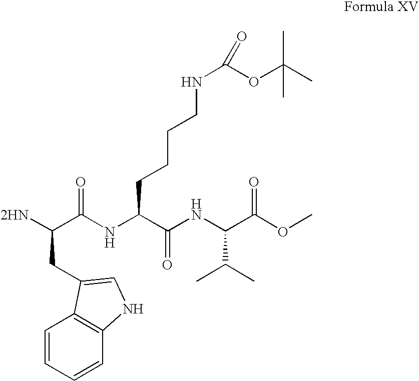

Step a) involves coupling of Val-OMe of Formula IX or a salt thereof with Z-Lys(Boc)-OH of Formula X to yield Z-Lys(Boc)-Val-OMe of Formula XI;

If Val-OMe of Formula IX is used in the form of salt, it is converted into the free base by treating with a suitable base.

Suitable bases, which can be used for neutralization of Formula IV, include but are not limited to N-methyl morpholine, ammonia and the like.

The quantity of base that can be used in the neutralization is about 2 to about 5 molar equivalents, per equivalent of Val-OMe of Formula IX.

The compound Z-Lys(Boc)-Val-OMe of Formula XI is produced by the formation of a mixed anhydride with chloroformate.

Suitable chloroformates, which can be used in the formation of a mixed anhydride, include but are not limited to methyl chloroformate, ethyl chloroformate, isobutylchloroformate and the like.

Suitable temperatures for conducting the reaction range from about −30° C. to about 100° C., or about −10° C. to about 30° C.

After completion of the reaction, the reaction mixture is filtered and the filtrate is washed with an acid solution, then with a base solution, followed by sodium chloride solution (brine solution) to remove byproducts. These acids and bases can be used in the form of aqueous solutions.

Acids that can be used in the washing include but are not limited to hydrochloric acid, hydrobromic acid, sulphuric acid and the like.

Bases that can be used in the washing include but are not limited to sodium bicarbonate, potassium carbonate, sodium carbonate, potassium bicarbonate and the like.

The concentration of acid that can be used in the above reaction is 0.1 to about 1N.

The concentration of the base, which can be used in the above reaction, is about 5% to about 15%.

The organic solution comprising Z-Lys(Boc)-Val-OMe of Formula XI may be used directly in the next processing step or it can be concentrated completely to form a solid.

Step b) involves converting Z-Lys(Boc)-Val-OMe of Formula XI to H-Lys(Boc)-Val-OMe of Formula XII;

Suitable solvents that can be used in step b) include but are not limited to: dichloromethane, chloroform and the like; ethers such as THF, diethyl ether, diisopropyl ether, 1,4-dioxane and the like; esters such as ethyl acetate, methyl acetate, and the like; and aromatic solvents such as benzene, toluene, and the like.

Z-Lys(Boc)-Val-OMe of Formula XI is treated with an acid and a deprotecting reagent in the presence of hydrogen gas.

The hydrogen gas pressure that can be used in the above reaction is to about 40 to about 50 psi (2.5 to 4 kg/cm²).

Suitable reagents that can be used for deprotection in the above reaction include but are not limited to catalysts such as palladium on carbon, palladium hydroxide, nickel, platinum, lithium aluminum hydride and the like.

Suitable acids which can be used in the above reaction include but are not limited to acetic acid, triflouroacetic acid, and the like.

After completion of the reaction, the reaction mixture may be filtered through a medium such as a celite bed to remove catalyst. The product is isolated from the filtrate by using suitable isolation techniques such as distillation.

Step c) involves coupling of H-Lys(Boc)-Val-OMe of Formula XII with Z-(D)-Trp-OH of Formula XIII to yield Z-(D) Trp-Lys(Boc)-Val-OMe of Formula XIV.

The compound Z-(D) Trp-Lys(Boc)-Val-OMe of Formula XIV is produced by the formation of a mixed anhydride with alkyl chloroformate.

Suitable alkyl chloroformates, which can be used in the formation of a mixed anhydride, include but are not limited to methyl chloroformate, ethyl chloroformate, Isobutylchloroformate and the like.

If H-Lys(Boc)-Val-OMe of Formula XII and Z-(D)-Trp-OH of Formula XIII are used in the form of salts, are converted into free bases by treating with a base.

Suitable bases, which can be used for neutralization of peptide moieties, include but are not limited to N-methyl morpholine, ammonia, sodium bicarbonate, sodium carbonate, potassium bicarbonate, potassium carbonate and the like.

The quantity of base that can be used in the neutralization is about 2 to about 5 molar equivalents, per equivalent of H-Lys(Boc)-Val-OMe of Formula XII and Z-(D)-Trp-OH of Formula XIII.

Suitable temperatures for conducting the reaction range from about −30° C. to about 100° C., or about −10° C. to about 30° C.

After completion of the reaction, the reaction mixture is filtered and washed with acid solution and base solution to remove byproducts.

Acidic solutions that can be used for washing is prepared by the dissolution of acids such as hydrochloric acid, sulphuric acid in water.

The concentration of acid that can be used in the above reaction is to about 0.1 to about 1N.

Basic solutions that can be used in the above step are prepared by the dissolution of bases such as sodium bicarbonate, potassium carbonate, sodium carbonate, and potassium bicarbonate in water.

The concentration of the base, which can be used in the above reaction, is about 5% to about 15%.

These acids and bases can be used in the form of solid or in the form of aqueous or alcoholic solutions.

The organic solution comprising Z-(D) Trp-Lys(Boc)-Val-OMe of Formula XIV may be used directly in the next processing step or it can be concentrated completely to form a solid.

The formed solid comprising Z-(D) Trp-Lys(Boc)-Val-OMe of Formula XIV may be purified by using recrystalliztion or reslury techniques.

Suitable solvents which can be used in the purification of Formula XIV include but are not limited to: THF, 1,4-dioxane, diisopropyl ether, diethyl ether, ethyl acetate, methyl acetate, n-butyl acetate, acetonitrile, acetone, toluene, chlorinated solvents like dichloromethane (DCM), chloroform, dichloroethane, and the like; and mixtures of the above solvents.

Step d) involves converting Z-(D)-Trp-Lys(Boc)-Val-OMe of Formula XIV to H-(D) Trp-Lys(Boc)-Val-OMe of Formula XV.

Suitable solvents that can be used in step d) include but are not limited to water, alcoholic solvents such as methanol, ethanol, isopropyl alcohol, n-butanol and the like; chlorinated solvents such as dichloromethane, chloroform and the like; ethers such as THF, diethyl ether, diisopropyl ether, 1,4-dioxane and the like; esters such as ethyl acetate, methyl acetate, and the like; aromatic solvents such as benzene, toluene, and the like.

The compound of Z-(D) Trp-Lys(Boc)-Val-OMe of Formula XIV is reacted with an acid and deprotecing reagents in the presence of hydrogen gas.

The hydrogen gas pressure that can be used in the above reaction is about 20 to about 50 psi (2.5 to 4.0 kg/cm²).

Suitable deprotection reagents that can be used in the above reaction include but are not limited to palladium on carbon, palladium hydroxide, nickel, platinum, lithium aluminum hydride and the like.

Suitable acids which can be used in the above reaction include but are not limited to acetic acid, triflouroacetic acid, hydroxhloric acid, hydrobromic acid, p-toluene sulfonic acid and the like.

The concentration of acid that can be used in the above reaction is about 0.1 to about 1N.

Suitable temperatures for conducting the reaction range from about −30° C. to about 100° C., or about −10° C. to about 30° C.

These acids can be used in the form of solids or in the form of aqueous or alcoholic solutions.

After completion of the reaction, the reaction mixture is filtered, such as through a celite bed and washed with alcohol.

Alcohols, which can be used in the washing, include but are not limited to methanol, ethanol, isopropyl alcohol, n-butanol and the like.

The solution comprising H-(D)-Trp-Lys(Boc)-Val-OMe of Formula XV may be used directly in the next processing step or it can be concentrated completely to form a solid.

The coupling reagents which can be used in steps a) and c) include but are not limited to carbodiimide reagents like 1-[3-(dimethylamino)propyl]-3-ethylcarbodiimide hydrochloride (EDC), N-cyclohexyl-N′-isopropylcarbodiimide (CIC), N,N′-diisopropylcarbodiimide (DIC), and the like.

Suitable reagents which can be used in steps a) and c) to avoid racemization include but are not limited to 1-hydroxybenzotriazole hydrate, ethyl-1-hydroxy-1H-1,2,3-triazole-4-carboxylate (HOCt), and N-hydroxysuccinimide (HOSu); imidazloium reagents like 1,1′-carbonyldiimidazole (CDI), 1,1′-carbonylbis(3-methyl-imidazolium)triflate (CBMIT); 2-(benzotriazol-1-yl)oxy-1,3-dimethyl-imidazolidinium hexafluorophosphate (BOI), CMBI; mixed anhydride coupling reagents like Iso-butyl chloroformate (IBCF), ethyl chloroformate (ECF), Iso-propyl chloroformate (IPCF), PhO2CCl; uronium reagents like O-(benzotriazol-1-yl)-1,1,3,3-tetramethyluronium hexafluorophosphate (HBTU), O-benzotriazol-1-yl-1,1,3,3-tetramethyluronium tetrafluoroborate (TBTU), 2-succinimido-1,1,3,3-tetramethyluronium tetrafluoroborate (TSTU), 2-(5-norbornene-2,3-dicarboximido)-1,1,3,3-tetramethyluronium tetrafluoroborate (TNTU); phosphonium reagents like BOP, Bromotri (pyrrolidino) phosphonium hexafluorophosphate (PyBrop), Benzotriazol-1-yloxytri(pyrrolidino)phosphonium hexafluorophosphate (PyBOP), and Chlorotri(pyrrolidino)phosphonium hexafluorophosphate (PyClOP) and the like.

Suitable solvents that can be used in steps a) and c) include but are not limited to: ethers such diethyl ether, diisopropylether, tetrahydrofuran, 1,4-dioxane, and the like; esters such as ethyl acetate, methyl acetate, n-butyl acetate, and the like; chlorinated solvents such as dichloromethane, chloroform and the like; hydrocarbons such as hexane, heptane and the like; other solvents like dimethylformamide, dimethylsulphoxide, acetonitrile, acetone, and toluene; and combinations thereof. Bases, which can be used in the neutralization of peptides, include but are not limited to N-methylmorpholine, ammonia and the like.

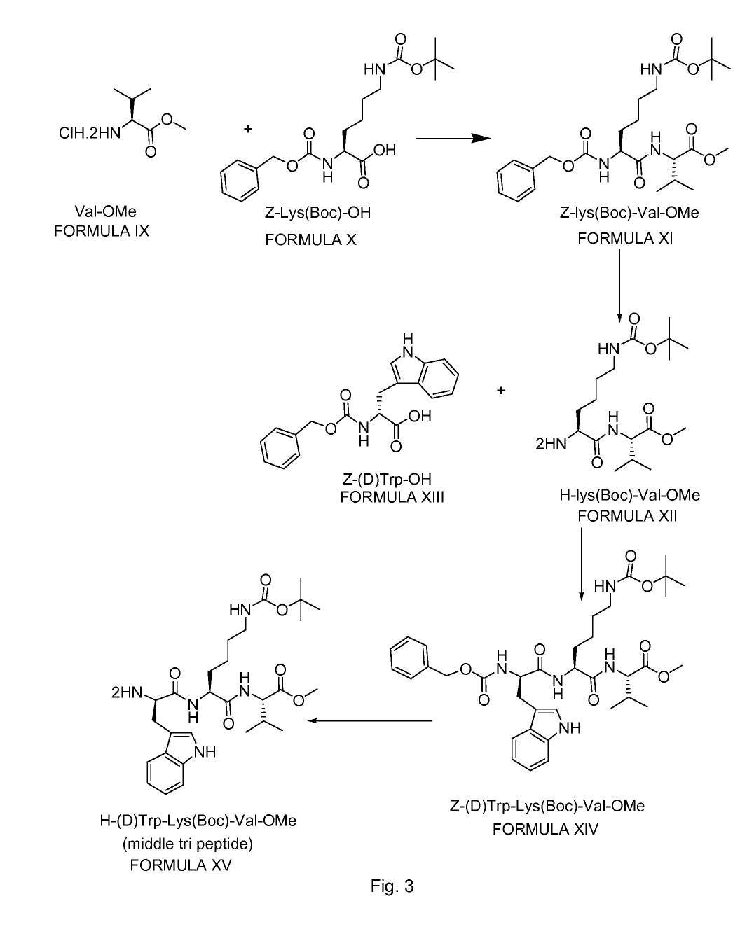

The above-mentioned process is schematically represented in FIG. 3.

In a fourth aspect, the invention provides a process for the preparation of H-Cys (PG)-Trp-NH₂ of Formula XXII, an intermediate used in the preparation of vapreotide, comprising:

a) reacting H-Trp-OH of Formula XVI with benzyloxy carbonyl chloride to yield Z-Trp-OH of Formula XVII;

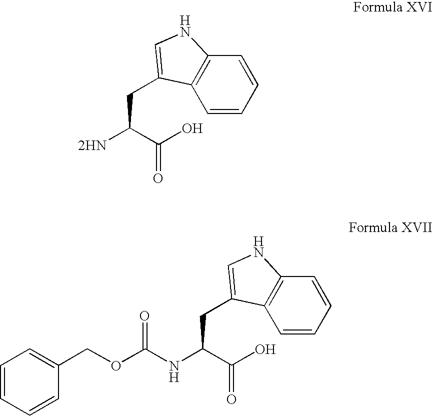

b) reacting Z-Trp-OH of Formula XVII with alkyl chloroformate; then with ammonia to yield Z-Trp-NH₂ of Formula XVIII;

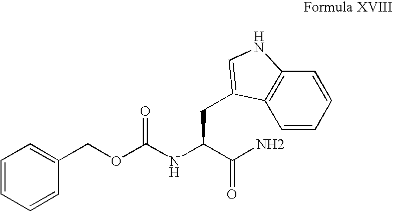

c) converting Z-Trp-NH₂ of Formula XVIII to H-Trp-NH₂ of Formula XIX;

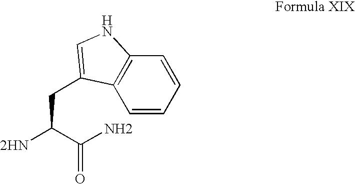

d) coupling of H-Trp-NH₂ of Formula XIX with Boc-Cys (PG)-OH of Formula XX to yield Boc-Cys (PG)-Trp-NH₂ of Formula XXI; and

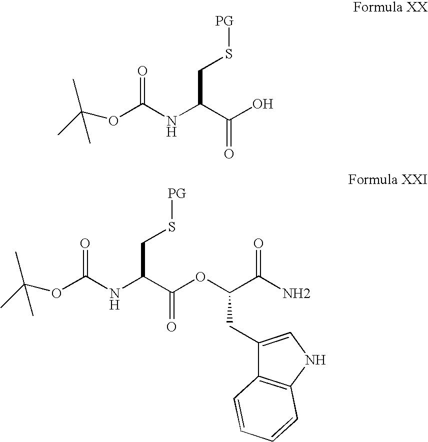

e) converting Boc-Cys (PG)-Trp-NH₂ of Formula XXI to H-Cys (PG)-Trp-NH₂ of Formula XXII in the presence of an acid.

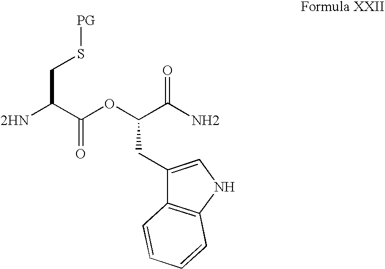

Step a) involves reacting H-Trp-OH of Formula XVI with benzyloxycarbonyl chloride in the presence of a suitable base to yield Z-Trp-OH of Formula XVII.

The concentration of benzyloxycarbonyl chloride which is used in step a) is to about 20% to about 50% in toluene.

Suitable bases that can be used in the formation of Formula XVII include but are not limited to N-methylmorpholine, ammonia, sodium carbonate, sodium bicarbonate, potassium carbonate, potassium bicarbonate, sodium hydroxide, potassium hydroxide, and the like.

After completion of the reaction, the reaction mixture is treated with an acid solution to obtain a pH of about 2 to about 6 to form the corresponding acid addition salt in form of a precipitate.

Suitable acid solutions that can be used in the above reaction include but are not limited to hydrochloric acid, sulphuric acid and the like; in the presence of water.

The concentration of acid that can be used in the above reaction is about 0.1 to about 1N in water.

Suitable temperatures for conducting the reaction range from about −30° C. to about 100° C., or about −10° C. to about 30° C.

The solid can be isolated by conventional techniques such as filtering, decanting, centrifuging and the like, or by filtering under an inert atmosphere using gases such as for example nitrogen and the like.

The wet cake obtained may optionally be further dried. Drying can be suitably carried out in a tray dryer, vacuum oven, air oven, fluidized bed drier, spin flash dryer, flash dryer and the like. The drying can be carried out at various temperatures, depending on the solvent used, temperatures of about 35° C. to about 70° C. frequently being suitable. The drying can be carried out for any desired time period until a desired product purity is obtained. Useful drying times frequently are from about 1 to 20 hours, or longer.

Step b) involves reacting Z-Trp-OH of Formula XVII with an alkyl chloroformate; then with ammonia to yield Z-Trp-NH₂ of Formula XVIII.

If Z-Trp-OH of Formula XVII is used in the form of salt, it is converted to the free base by treating with a suitable base.

Suitable bases, which can be used for neutralization of salt, include but are not limited to N-methyl morpholine, ammonia, sodium carbonate, sodium bicarbonate, potassium carbonate, potassium bicarbonate, sodium hydroxide, potassium hydroxide, and the like.

The quantity of base that can be used in the neutralization is about 2-5 equivalents, per equivalent of Z-Trp-OH of Formula XVII.

Suitable temperatures for conducting the reaction range from about −30° C. to about 100° C., or about −20° C. to about 20° C.

Suitable solvents that can be used in the above reaction include but are not limited to: esters such as ethyl acetate, methyl acetate, isobutyl ester, ethylacetoacetate and the like; and chlorinated solvents such as dichloromethane, chloroform and the like.

The solid can be isolated by conventional techniques such as filtering, decanting, centrifuging and the like, or by filtering under an inert atmosphere using gases such as for example nitrogen and the like.

The wet cake obtained may optionally be further dried. Drying can be suitably carried out in a tray dryer, vacuum oven, air oven, fluidized bed drier, spin flash dryer, flash dryer and the like. The drying can be carried out at various temperatures, such as about 35° C. to about 70° C. The drying can be carried out for any desired time periods until a desired product purity is obtained. Useful drying times frequently are from about 1 to 20 hours, or longer.

Step c) involves converting Z-Trp-NH₂ of Formula XVIII to H-Trp-NH₂ of Formula XIX.

Suitable solvents that can be used in the reduction of step e) include but are not limited to: dichloromethane, chloroform and the like; ethers such as THF, diethyl ether, diisopropyl ether, 1,4-dioxane and the like; esters such as ethyl acetate, methyl acetate, and the like; aromatic solvents such as benzene, toluene, and the like;

Suitable reagents that can be used in step c) include but are not limited to palladium on carbon, palladium hydroxide, nickel, platinum in the presence of hydrogen or lithium aluminum hydride, and the like.

Suitable temperatures for conducting the reaction range from about 10° C. to about 100° C., or about 30° C. to about 50° C.

After completion of the reaction, the reaction mixture is filtered, such as through a Hyflow (flux calcined diatomaceous earth) bed, and washed with an alcohol like methanol, ethanol, isopropyl alcohol or mixtures thereof.

Step d) involves coupling of H-Trp-NH₂ of Formula XIX with Boc-Cys (PG)-OH of Formula XX to yield Boc-Cys (PG)-Trp-NH₂ of Formula XXI.

If H-Trp-NH₂ of Formula XIX and Boc-Cys (PG)-OH of Formula XX are used in the form of salts, they are converted to the free base by treating with a suitable base.

Suitable bases, which can be used for neutralization of Boc-Cys (PG)-Trp-NH₂ of Formula XXI, include but are not limited to N-methyl morpholine, ammonia, sodium carbonate, sodium bicarbonate, potassium carbonate, potassium bicarbonate, sodium hydroxide, potassium hydroxide, and the like.

The quantity of base that can be used in the neutralization is about 2 to about 5 molar equivalents, per equivalent of H-Trp-NH₂ of Formula XIX.

Suitable temperatures for conducting the reaction range from about −30° C. to about 100° C., or about 0° C. to about 30° C.

After completion of the reaction, the reaction mixture is filtered to remove byproducts and the filtrate is concentrated to form a residue. The residue is dissolved in a suitable solvent and washed with aqueous acid and base solutions.

Suitable solvents which can be used for dissolving the residue include but are not limited to: water; esters such as ethyl acetate, methyl acetate, n-butyl acetate and the like; and ethers such as diethyl ether, diisopropyl ether, THF, 1,4-dioxane and the like.

Acidic solutions that can be used in the washing are prepared by the dissolution of acids such as hydrochloric acid, sulphuric acid in water.

The concentration of acid that can be used in the washing is to about 0.1 to about 2N.

Basic solutions that can be used in the washing are prepared by the dissolution of bases such as sodium bicarbonate, potassium carbonate, sodium carbonate, and potassium bicarbonate, sodium hydroxide, potassium hydroxide in water.

The concentration of base that can be used in the washing is to about 0.1 to about 2N.

Step e) involves converting Boc-Cys (PG)-Trp-NH₂ of Formula XXI to H-Cys (PG)-Trp-NH₂ of Formula XXII in the presence of an acid.

Suitable acids which can be used in step e) include but are not limited to TFA, acetic acid, hydrochloric acid, hydrobromic acid, p-toluene sulphonic acid, and the like; in the presence of thioanisole.

Suitable solvents that can be used in the step of e) include but are not limited to: dichloromethane, chloroform and the like; ethers such as THF, diethyl ether, diisopropyl ether, 1,4-dioxane and the like; esters such as ethyl acetate, methyl acetate, and the like; aromatic solvents such as benzene, toluene, and the like;

If Boc-Cys (PG)-Trp-NH₂ is used in the form of salt, it is converted to the free base by treating with a suitable base. Suitable bases are not limited to N-methyl morpholine, ammonia, sodium bicarbonate, potassium carbonate, sodium carbonate, and potassium bicarbonate, sodium hydroxide, potassium hydroxide and the like.

These bases can be used in the form of solids or in the form of aqueous or alcoholic solutions.

The coupling reagents which can be used in step d) include but are not limited to carbodiimide reagents like 1-[3-(dimethylamino)propyl]-3-ethylcarbodiimide hydrochloride (EDC), N-cyclohexyl-N′-isopropylcarbodiimide (CIC), N,N′-diisopropylcarbodiimide (DIC), and the like.

Suitable reagents which can be used in step d) to avoid racemization include but are not limited to 1-hydroxybenzotriazole hydrate, ethyl-1-hydroxy-1H-1,2,3-triazole-4-carboxylate (HOCt), and N-hydroxysuccinimide (HOSu); imidazloium reagents like 1,1′-carbonyldiimidazole (CDI), 1,1′-carbonylbis(3-methyl-imidazolium)triflate (CBMIT); 2-(benzotriazol-1-yl)oxy-1,3-dimethyl-imidazolidinium hexafluorophosphate (BOI), CMBI; mixed anhydride coupling reagents like Iso-butyl chloroformate (IBCF), ethyl chloroformate (ECF), Iso-propyl chloroformate (IPCF), PhO2CCl; uronium reagents like O-(benzotriazol-1-yl)-1,1,3,3-tetramethyluronium hexafluorophosphate (HBTU), O-benzotriazol-1-yl-1,1,3,3-tetramethyluronium tetrafluoroborate (TBTU), 2-succinimido-1,1,3,3-tetramethyluronium tetrafluoroborate (TSTU), 2-(5-norbornene-2,3-dicarboximido)-1,1,3,3-tetramethyluronium tetrafluoroborate (TNTU); phosphonium reagents like BOP, Bromotri (pyrrolidino) phosphonium hexafluorophosphate (PyBrop), benzotriazol-1-yloxytri(pyrrolidino)phosphonium hexafluorophosphate (PyBOP), and chlorotri(pyrrolidino)phosphonium hexafluorophosphate (PyClOP) and the like.

Suitable solvents that can be used in step d) include but are not limited to: ethers such diethyl ether, diisopropylether, tetrahydrofuran, 1,4-dioxane, and the like; esters such as ethyl acetate, methyl acetate, n-butyl acetate, and the like; chlorinated solvents such as dichloromethane, chloroform and the like; hydrocarbons such as hexane, heptane and the like; other solvents like dimethylformamide, dimethylsulphoxide, acetonitrile, acetone, and toluene; and combinations thereof.

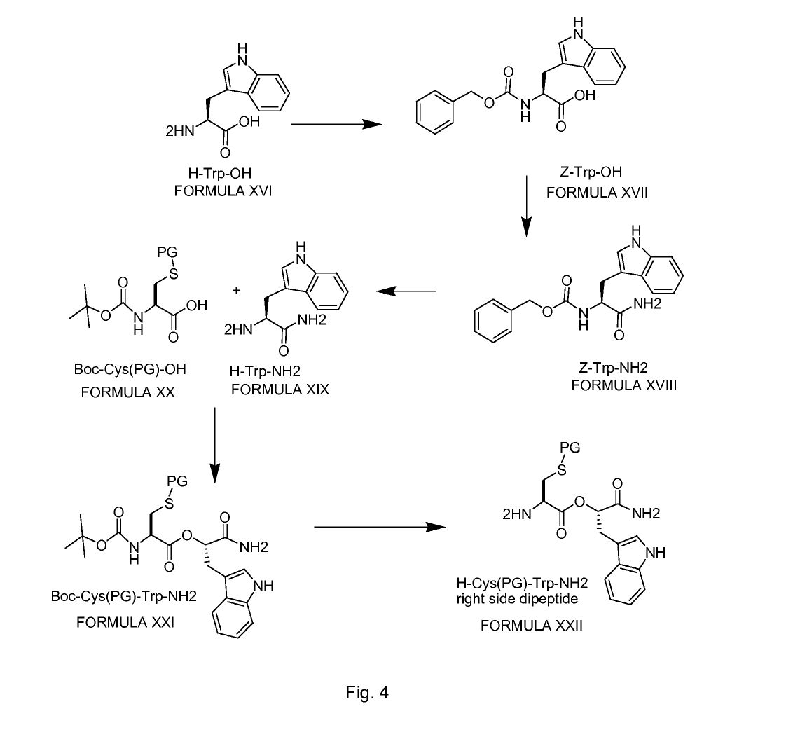

The above-described process is schematically represented in FIG. 4.

In a fifth aspect, the present invention provides another process for the preparation of vapreotide of Formula I, and its pharmaceutically acceptable salts. A process for preparing vapreotide comprises:

a) conversion of H (D) Trp-Lys(Boc)-Val-OMe of Formula XXVII in the presence of a base to yield H (D) Trp-Lys(Boc)-Val-OH of Formula XXVIII;

b) conversion of H (D) Trp-Lys(Boc)-Val-OH of Formula XXVIII to Fmoc-(D)-Trp-Lys(Boc)-Val-OH of Formula XXIX using Fmoc.Cl in the presence of a base;

c) coupling of Fmoc-(D)-Trp-Lys(Boc)-Val-OH of Formula XXIX with TFA-Cys (Acm)-Trp-NH₂ to yield Fmoc-(D)-Trp-Lys(Boc)-Val-Cys (Acm)-Trp-NH₂ of Formula XXX;

d) conversion of Fmoc-(D)-Trp-Lys(Boc)-Val-Cys(Acm)-Trp-NH₂ in the presence of a base to yield H-(D)-Trp-Lys(Boc)-Val-Cys(Acm)-Trp-NH₂ of Formula XXXI;

e) coupling of H-(D)-Trp-Lys(Boc)-Val-Cys (Acm)-Trp-NH₂ of Formula XXXI with Boc-(D)-Phe-Cys (Acm)-Tyr-OH to yield Boc-(D)-Phe-Cys (Acm)-Tyr-(D)-Trp-Lys(Boc)-Val-Cys (Acm)-Trp-NH₂ of Formula XXXII;

f) conversion of Boc-(D)-Phe-Cys(Acm)-Tyr-(D)-Trp-Lys(BOC)-Val-Cys(Acm)-Trp-NH₂ of Formula XXXII to NH₂-(D)Phe-Cys(Acm)-Tyr-(D)-Trp-Lys-Val-Cys(Acm)-Trp-NH₂ of Formula XXXIII; and

g) conversion of NH₂-(D) Phe-Cys (Acm)-Tyr-(D)-Trp-Lys-Val-Cys (Acm)-Trp-NH₂ of Formula XXXIII to vapreotide of Formula I, or a salt thereof.

Step a) involves conversion of H (D) Trp-Lys(Boc)-Val-OMe of Formula XXVII in the presence of a base to yield H (D) Trp-Lys(Boc)-Val-OH of Formula XXVIII.

Suitable bases which can be used in the formation of H (D) Trp-Lys(Boc)-Val-OH of Formula XXVIII include but are not limited to sodium hydroxide, potassium hydroxide, sodium bicarbonate, potassium bicarbonate, sodium carbonate, potassium carbonate and the like.

Suitable solvents that can be used in the formation of Formula XXVIII include but are not limited to: alcoholic solvents such as methanol, ethanol, isopropyl alcohol, and the like; ethers such as diethyl ether, diisopropyl ether, THF, 1,4-dioxane and the like; other solvents such as water; and the like.

Suitable temperatures for conducting the reaction range from about −20° C. to about 100° C., or about −5° C. to about 30° C.

After completion of the reaction, the reaction mixture is acidified with an acid to get a pH about 1 to about 3 and is extracted into an organic solvent.

Suitable acids that can be used include but are not limited to hydrochloric acid, hydrobromic acid, sulphuric acid, nitric acid, phosphoric acid, and the like.

The concentration of the acid that can be used in the above reaction is about 0.1 to about 0.5 N in water.

Suitable organic solvents that can be used in the extraction of the compound of Formula XXVIII include but are not limited to: alcohols such as methanol, ethanol, isopropyl alcohol, n-butanol, and the like; esters such as ethyl acetate, methyl acetate, ethyl acetoacetate, n-butyl acetate and the like; and chlorinated solvents such as dichloromethane, chloroform and the like.

The organic solution comprising H-(D)Trp-Lys(Boc)-Val-OH of Formula XXVIII may be used directly in the next processing step or it can be concentrated completely to form a solid.

Step b) involves conversion of H-(D)Trp-Lys(Boc)-Val-OH of Formula XXVIII to Fmoc-(D)-Trp-Lys(Boc)-Val-OH of Formula XXIX using Fmoc.Cl in the presence of a base.

The quantity of Fmoc.Cl, which can be used in the preparation of Formula XXIX, is to about 1 equivalent, per equivalent of H (D) Trp-Lys(Boc)-Val-OH of Formula XXVIII.

Suitable bases which can be used in the above reaction include but are not limited to sodium carbonate, potassium carbonate, sodium hydroxide, potassium hydroxide, sodium bicarbonate, potassium bicarbonate, N-methylmorpholine, ammonia and the like.

The quantity of base used in the formation of Formula XXIX is to about 1 to about 5 molar equivalents, per equivalent of H (D) Trp-Lys(Boc)-Val-OH of Formula XXVIII.

Suitable solvents which can be used in the above reaction include but are not limited to: water; ethers such as diethyl ether, diisopropyl ether, THF, 1,4-dioxane and the like; and alcohols such as methanol, ethanol, isopropyl alcohol, n-butanol and the like.

Suitable temperatures for conducting the reaction range from about −20° C. to about 100° C., or about −5° C. to about 25° C.

After completion of the reaction, the reaction mixture is diluted with water, acidified to get a pH about 1 to about 3 and Fmoc-(D)-Trp-Lys(Boc)-Val-OH of Formula XXIX is extracted into an organic solvent.

Suitable acids that can be used in the acidification include but are not limited to hydrochloric acid, hydrobromic acid, sulphuric acid, nitric acid, phosphoric acid, and the like.

The concentration of the acid that can be used in the above reaction is about 0.1 to about 0.5 N in water.

Suitable organic solvents that can be used in the extraction of the compound of Formula XXVIII include but are not limited to: alcohols such as methanol, ethanol, isopropyl alcohol, n-butanol, and the like; esters such as ethyl acetate, methyl acetate, ethyl acetoacetate, n-butyl acetate and the like; and chlorinated solvents such as dichloromethane, chloroform and the like.

Step c) involves coupling of Fmoc-(D)-Trp-Lys(Boc)-Val-OH of Formula XXIX with TFA-Cys (Acm)-Trp-NH₂ to yield Fmoc-(D)-Trp-Lys(Boc)-Val-Cys (Acm)-Trp-NH₂ of Formula XXX.

TFA-Cys (Acm)-Trp-NH₂, which is used in the preparation of Fmoc-(D)-Trp-Lys(Boc)-Val-Cys (Acm)-Trp-NH₂ of Formula XXX, is prepared from Boc-Cys (Acm)-Trp-NH₂ in the presence of an acid.

Suitable acids that can be used in the preparation of TFA-Cys (Acm)-Trp-NH₂ include but are not limited to hydrochloric acid, hydrobromic acid, sulphuric acid, nitric acid, phosphoric acid, and the like.

Suitable solvents which can be used in the preparation of TFA-Cys (Acm)-Trp-NH₂ include but are not limited to: chlorinated solvents such as dichloromethane, chloroform and the like; ethers such as diethyl ether, diisopropyl ether, THF, 1,4-dioxane and the like; alcohols such as methanol, ethanol, isopropyl alcohol, n-butanol and the like; and esters such as ethyl acetate, methyl acetate, ethyl acetoacetae, methyl acetoacetate, n-butyl acetate, and the like.

Suitable temperatures for conducting the reaction range from about −20° C. to about 100° C., or about −5° C. to about 25° C.

After completion of the reaction, the reaction mixture is concentrated completely, triturated with ether and the solvent decanted from the residue.

TFA-Cys (Acm)-Trp-NH₂ of Formula XXX is used in the form of a salt, so it can be converted to the free base by treating with a suitable base in the presence of a suitable solvent.

Suitable bases which can be used in the neutralization include but are not limited to N-methyl morpholine, ammonia, sodium carbonate, potassium carbonate, sodium hydroxide, potassium hydroxide, sodium bicarbonate, potassium bicarbonate and the like.

Suitable solvents that can be used in the neutralization include but are not limited to: DMF, DMSO, dimethyl acetamide (DMA) and the like; and alcohols such as methanol, ethanol, isopropyl alcohol, n-butanol and the like.

The formed solution comprising H-Cys (Acm)-Trp-NH₂ is coupled with Fmoc-(D)-Trp-Lys(Boc)-Val-OH of Formula XXIX to form Fmoc-(D)-Trp-Lys(Boc)-Val-Cys (Acm)-Trp-NH₂ of Formula XXX.

Suitable temperatures for conducting the reaction range from about −20° C. to about 100° C., or about −5° C. to about 25° C.

After completion of the reaction, the reaction mixture is diluted with organic solvent and the formed solid isolated.

The solid isolation can be conducted by conventional techniques such as filtering, decanting, centrifuging and the like, or by filtering under an inert atmosphere using gases such as for example nitrogen and the like.

Suitable organic solvents which can be used in the dilution include but are not limited to esters such as ethyl acetate, methyl acetate, n-butyl acetate, ethyl acetoacetate, methyl acetoacetate and the like.

Step d) involves conversion of Fmoc-(D)-Trp-Lys(Boc)-Val-Cys(Acm)-Trp-NH₂ in the presence of a base to yield H-(D)-Trp-Lys(Boc)-Val-Cys(Acm)-Trp-NH₂ of Formula XXXI.

Suitable bases which can be used in the formation of H-(D)-Trp-Lys(Boc)-Val-Cys(Acm)-Trp-NH₂ of Formula XXXI include but are not limited to ammonia, diethylamine, triethyl amine, isopropyl amine, dimethyl amine and the like.

Suitable solvents which can be used in the formation of H-(D)-Trp-Lys(Boc)-Val-Cys(Acm)-Trp-NH₂ of Formula XXXI include but are not limited to: hydrocarbons such as n-hexane, cyclohexane, n-heptane, toluene, xylenes and the like; aprotic polar solvents such as N,N-dimethyl formamide (DMF), dimethylsulfoxide, dimethylacetamide, and the like; nitriles such as acetonitrile, 2-propenenitrile, cyclopentanecarbonitrile and the like; ketones such as acetone, methyl isobutyl ketone, methyl tertiary butyl ketone, and the like; and mixtures thereof.

Suitable temperatures for conducting the reaction range from about 0° C. to about 100° C., or about 20° C. to about 35° C.

After completion of the reaction, the reaction mixture is concentrated completely and purified by using recrystallization or reslury techniques.

Suitable solvents that can be used in the above purification include but are not limited to ethers such as diethyl ether, diisopropyl ether, dimethyl ether, THF, 1,4-dioxane and the like.

The reaction solution comprising H-(D)-Trp-Lys(Boc)-Val-Cys(Acm)-Trp-NH₂ of Formula XXXI may be used directly in the next processing step or it can be isolated to get a solid.

The solid can be separated by concentrating a solution, or precipitation by adding antisolvent or by recrystallization.

The solid isolation can be conducted by conventional techniques such as filtering, decanting, centrifuging, and the like, or by filtering under an inert atmosphere using gases such as for example nitrogen and the like.

Step e) involves coupling of H-(D)-Trp-Lys(Boc)-Val-Cys (Acm)-Trp-NH₂ of Formula XXXI with Boc-(D)-Phe-Cys (Acm)-Tyr-OH to yield Boc-(D)-Phe-Cys (Acm)-Tyr-(D)-Trp-Lys(Boc)-Val-Cys (Acm)-Trp-NH₂ of Formula XXXII.

If H-(D)-Trp-Lys(Boc)-Val-Cys (Acm)-Trp-NH₂ of Formula XXXI and Boc-(D)-Phe-Cys (Acm)-Tyr-OH are used in the form of salts, these are converted to the free base by the treatment with a suitable base.

Suitable temperatures for conducting the reaction range from about −30° C. to about 100° C., or about 0° C. to about 35° C.

After completion of the reaction, the reaction mixture is diluted with organic solvent and filtered to remove the byproduct.

Suitable solvents that can be used for the dilution include but are not limited to: esters such as ethyl acetate, methyl acetate, ethyl acetoacetate, n-butyl acetate and the like; chlorinated solvents such as dichloromethane, chloroform and the like; and hydrocarbons such as n-hexane, heptane, cyclohexane and the like.

The obtained filtrate is distilled to removed the solvent and the product is isolated.

Step f) involves conversion of Boc-(D)-Phe-Cys(Acm)-Tyr-(D)-Trp-Lys(BOC)-Val-Cys(Acm)-Trp-NH₂ of Formula XXXII to NH₂-(D)Phe-Cys(Acm)-Tyr-(D)-Trp-Lys-Val-Cys(Acm)-Trp-NH₂ of Formula XXXIII, or a salt thereof.

Suitable acids that can be used in the formation of Formula XXXIII include but are not limited to trifloroacetic acid, hydrochloric acid , benzene sulfonic acid and the like; in the presence of thioanisole.

Suitable solvents that can be used in step f) include but are not limited to: dichloromethane, chloroform and the like; ethers such as THF, diethyl ether, diisopropyl ether, 1,4-dioxane and the like; esters such as ethyl acetate, methyl acetate, and the like; aromatic solvents such as benzene, toluene, and the like;

Suitable temperatures for conducting the reaction range from about −30° C. to about 100° C., or about −10° C. to about 30° C.

Suitable salts which can be formed in the above reaction include but are not limited to the hydrochloride salt, trifluoroacetate salt, mesylate salt, besylate salt and the like.

The compound of NH₂-(D)Phe-Cys(Acm)-Tyr-(D)-Trp-Lys-Val-Cys(Acm)-Trp-NH₂ of Formula XXXIII or its salt may be purified, using recrystallization or reslury techniques.

Suitable solvents that can be used in the purification include but are not limited to alcoholic solvents such as methanol, ethanol, isopropyl alcohol and the like, or mixtures of solvents.

Step g) involves conversion of NH₂-(D) Phe-Cys (Acm)-Tyr-(D)-Trp-Lys-Val-Cys (Acm)-Trp-NH₂ of Formula XXXIII to vapreotide of Formula I, or a salt thereof.

Suitable acids that can be used in the formation of vapreotide of Formula I or salts thereof include but are not limited to acetic acid, trifluoroacetic acid, hydrochloric acid and the like, in the presence of iodine.

The concentration of the acid that can be used in step e) is in the range of about 0.01 to about 1% in water.

Suitable solvents that can be used in the step e) include but are not limited to water, methanol, ethanol, isopropyl alcohol, acetonitrile and the like; chlorinated solvents such as dichloromethane, chloroform and the like; ethers such as THF, diethyl ether, diisopropyl ether, 1,4-dioxane and the like; esters such as ethyl acetate, methyl acetate, and the like; aromatic solvents such as benzene, toluene, and the like.

Suitable temperatures for conducting the reaction range from about −30° C. to about 100° C., or about −10° C. to about 30° C.

After completion of the reaction, the reaction mass is quenched with sodium thiosulphate solution and purified using suitable purification techniques.

Suitable purification techniques that can be used in the above step include but are not limited to preparative high performance liquid chromatography, flash column chromatography, precipitation using antisolvent, and the like.

The purification of vapreotide can be achieved by recrystallization, reslury or a combination thereof, or preparative high performance liquid chromatography, or flash column chromatography, typically followed by drying of the resulting wet compound.

Suitable solvents which can be used in the purification include but are not limited to: water; alcoholic solvents such as methanol, ethanol, isopropyl alcohol, n-butanol and the like; nitriles such as acetonitrile, and the like; chlorinated solvents such as dichloromethane, chloroform and the like; and esters such as ethyl acetate, methyl acetate, butyl acetate and the like.

The solid can be isolated by conventional techniques such as filtering, decanting, centrifuging and the like, or by filtering under an inert atmosphere using gases such as for example nitrogen and the like.

The wet cake obtained may optionally be further dried. Drying can be suitably carried out in a tray dryer, vacuum oven, air oven, fluidized bed drier, spin flash dryer, flash dryer, freeze drier and the like. The drying can be carried out at temperatures of about 35° C. to about 70° C. The drying can be carried out for any desired time periods until the desired product purity is obtained; frequently times from about 1 to 20 hours, or longer, are used.

The coupling reagents which can be used in the steps c) and e) include but are not limited to carbodiimide reagents like 1-[3-(dimethylamino)propyl]-3-ethylcarbodiimide hydrochloride (EDC), N-cyclohexyl-N′-isopropylcarbodiimide (CIC), N,N′-diisopropylcarbodiimide (DIC), and the like.

Suitable reagents which can be used in the in steps c) and e) to avoid racemization include but are not limited to 1-hydroxybenzotriazole hydrate, ethyl-1-hydroxy-1H-1,2,3-triazole-4-carboxylate (HOCt), and N-hydroxysuccinimide (HOSu); imidazloium reagents like 1,1′-carbonyldiimidazole (CDI), 1,1′-carbonylbis(3-methyl-imidazolium)triflate (CBM IT); 2-(benzotriazol-1-yl)oxy-1,3-dimethyl-imidazolidinium hexafluorophosphate (BOI), CMBI; mixed anhydride coupling reagents like Iso-butyl chloroformate (IBCF), ethyl chloroformate (ECF), Iso-propyl chloroformate (IPCF), PhO2CCl; uronium reagents like O-(benzotriazol-1-yl)-1,1,3,3-tetramethyluronium hexafluorophosphate (HBTU), O-benzotriazol-1-yl-1,1,3,3-tetramethyluronium tetrafluoroborate (TBTU), 2-succinimido-1,1,3,3-tetramethyluronium tetrafluoroborate (TSTU), 2-(5-norbornene-2,3-dicarboximido)-1,1,3,3-tetramethyluronium tetrafluoroborate (TNTU); phosphonium reagents like BOP, Bromotri (pyrrolidino) phosphonium hexafluorophosphate (PyBrop), benzotriazol-1-yloxytri(pyrrolidino)phosphonium hexafluorophosphate (PyBOP), and chlorotri(pyrrolidino)phosphonium hexafluorophosphate (PyClOP) and the like.

Suitable solvents that can be used in the in the steps c) and e) include but are not limited to ethers such diethyl ether, diisopropylether, tetrahydrofuran, 1,4-Dioxane, and the like; esters such as ethyl acetate, methyl acetate, n-butyl acetate, and the like; chlorinated solvents such as dichloromethane, chloroform and the like; hydrocarbons such as hexane, heptane and the like; other solvents like dimethylformamide, dimethylsulphoxide, acetonitrile, acetone, and toluene; and combinations thereof. Bases, which can be used in the neutralization of peptides, include but are not limited to N-methylmorpholine, ammonia and the like.

The process is depicted in the following reaction scheme:

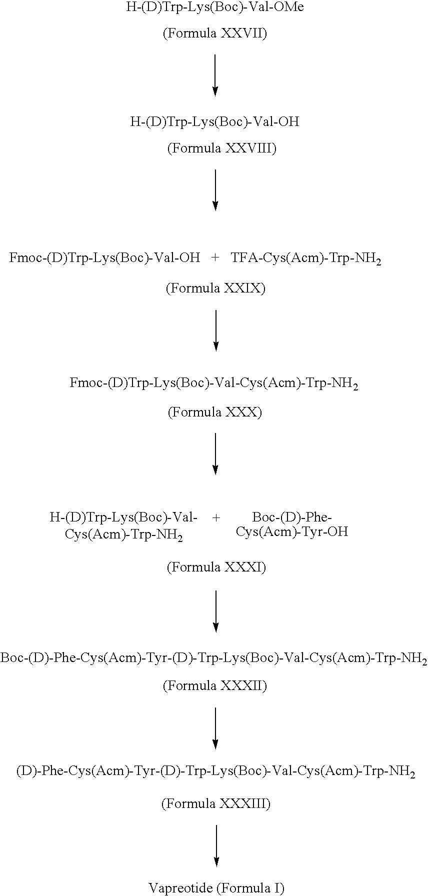

In a sixth aspect, the invention provides a compound H-Lys(Boc)-Val-OMe of Formula XII and a process for its preparation.

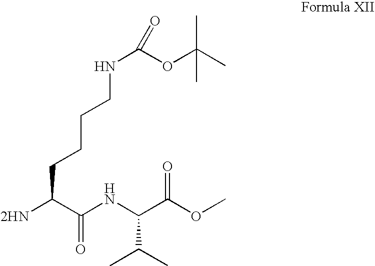

In a seventh aspect, the invention provides a compound Z-(D) Trp-Lys(Boc)-Val-OMe of Formula XIV, where Z represents a benzyloxy carbonyl group and a process for its preparation.

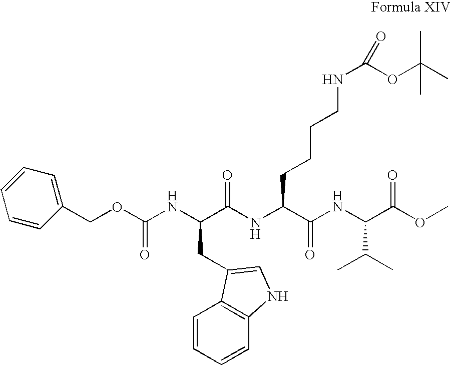

In an eighth aspect, the invention provides a compound Boc-(D) Phe-Cys (Acm)-Tyr-OH of Formula VIII, and a process for its preparation.

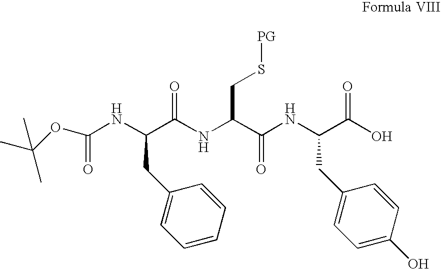

In a ninth aspect, the invention provides a compound Boc-(D) Phe-Cys(Acm)-Tyr-(D) Trp-Lys(Boc)-Val-Cys(Acm)-Trp-NH₂of Formula XXV and a process for its preparation.

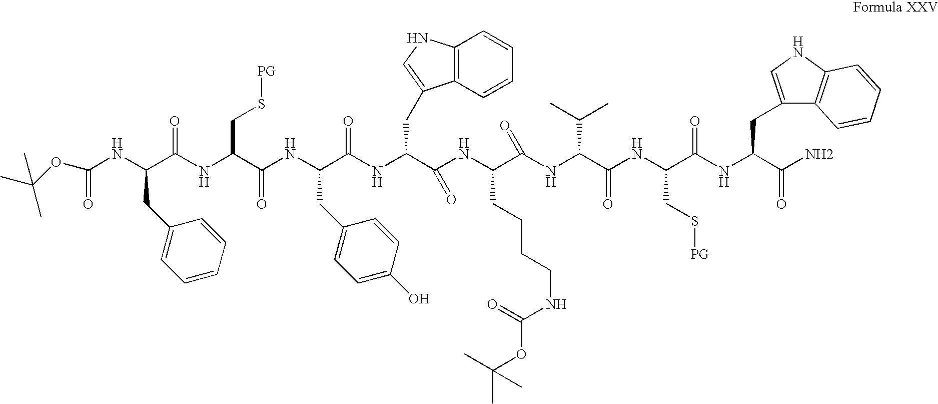

It has been surprisingly found that solution phase synthesis is advantageous for the preparation of vapreotide of Formula I, and its pharmaceutically acceptable salts.

Certain specific aspects and embodiments of the invention are further illustrated by the following examples, which are not intended to limit the scope of the invention.

### EXAMPLE 1

### Preparation of Boc-Cys (Acm) Tyr-OMe (Formula IV)

50 g of Boc-Cys (Acm)-OH of Formula II was taken into a round bottom flask and 150 ml of tetrahydrofuran was added. In another flask 43.6 g of H-Tyr-OMe Hydrochloride salt of Formula III and 100 ml of tetrahydrofuran were taken. 21.1 ml of N-methyl morpholine was added to the reaction mixture at 10° C. and stirred for 15 minutes. The suspension of H-Tyr-OMe hydrochloride in tetrahydrofuran was added to the solution of Boc-Cys (Acm)-OH in tetrahydrofuran at 0-10° C. 25.4 g of 1-hydroxybenzotriazole hydrate was added to the reaction in a single lot and the reaction mixture was cooled to 0 to 5° C. 38.8 g of 1,3 dicyclohexylcarbodiimide was added to the reaction at 0 to 5° C. and stirred at the same temperature for 1 hour. The reaction temperature was brought to room temperature and continued for 14 hours. Confirmed the reaction completion by thin layer chromatography (TLC). After reaction completion the reaction mass was cooled to −5 to −10° C. and stirred for 1 hour and then the precipitated dicyclohexylurea was filtered and the cake was washed with 10 ml of cold tetrahydrofuran. The tetrahydrofuran was concentrated completely under vacuum below 40° C. and the residue was dissolved in 500 ml of ethyl acetate. The ethyl acetate solution was washed successively with 200 ml of 0.5 N hydrochloric acid twice, 100 ml of water, 200 ml of 10% aqueous sodium bicarbonate solution twice, and finally with 300 ml of water three times. The organic layer was dried over sodium sulphate, filtered and concentrated below 40° C. under vacuum to get 72 g of light brown colored solid of title compound.

### EXAMPLE 2

### Preparation of Boc-(D)-Phe-Cys (Acm)-Tyr-OMe (Formula VII)

80 g of Boc-Cys (Acm)-Tyr-OMe of Formula IV was dissolved in 120 ml of dichloromethane and 120 ml of trifluroacetic acid was added dropwise to the reaction mass at 25 to 35° C. Stirred the reaction mass at 25 to 35° C. for 40 minutes. Confirmed the reaction completion by TLC. After reaction completion the reaction mass was concentrated at 45° C. under high vacuum to get a residue. The obtained residue of H-Cys (Acm)-Tyr-OMe of Formula V was dissolved in 200 ml of tetrahydrofuran, cooled to 0 to 5° C., and the pH was adjusted to 8 with 72 ml of N-methyl morpholine.

34.7 g of Boc-(D)-Phe-OH of Formula VI, 100 ml of tetrahydrofuran and 21.7 ml of N-methyl morpholine were charged in a flask and cooled to 0 to 5° C. 12.7 ml of isobutylchloroformate (IBCF) was added dropwise for 15 minutes at the same temperature. The reaction was stirred further for 10 minutes and then neutralized with N-methylmorpholine. H-Cys (Acm)-Tyr-OMe solution (prepared above) was added dropwise to the reaction mass over 30 minutes at 0-5° C. The reaction temperature was brought to room temperature and continued for 3 hours. Confirmed the reaction completion by high performance liquid chromatography. After reaction completion 200 ml of water, and 600 ml of ethyl acetate were added to the reaction mass and stirred for 15 minutes. The organic layer was separated and washed successively with 500 ml of 0.5 N hydrochloric acid twice, 500 ml of water, 500 ml of 10% sodium bicarbonate solution twice, and finally with 500 ml of water. The organic layer was dried over sodium sulphate, filtered, and distilled completely at 40° C. to give a yellow colored fluffy solid. The solid was dissolved in 300 ml of ethyl acetate and 200 ml of a solution of petroleum ether and diisopropylether (ratio is 1:1) was slowly added. The obtained reaction suspension was stirred for 8 hours at 25-35° C. and filtered to get 45 g of the title compound.

### EXAMPLE 3

### Preparation of Boc-(D)Phe-Cys(Acm)Tyr-OH (Formula VIII)

42 g of Boc-(D)-Phe-Cys (Acm)-Tyr-OMe of Formula VII was dissolved in 336 ml methanol, and the solution was cooled to 0 to 10° C. 408.6 ml of aqueous 1 N sodium hydroxide solution was added dropwise to the reaction over 40 minutes at 0 to 10° C. and continued at the same temperature for 2 hours. Confirmed the reaction completion by TLC. After reaction completion the reaction mass was diluted with 400 ml of water and washed with 200 ml of ethyl acetate twice. Separated the aqueous layer. The pH of the aqueous layer was adjusted to 3.5 with saturated aqueous potassium hydrogen sulphate (KHSO₄) and extracted with 400 ml ethyl acetate. The organic layer was washed with 200 ml of water twice and dried over sodium sulphate. The organic layer was filtered and concentrated at 35° C. using vacuum to give a sticky mass. The sticky mass was stirred with 400 ml diisopropyl ether for 6 hours at 25 to 35° C. and a white solid product formed. Filtered the product to get 37 g of the title compound.

### EXAMPLE 4

### Preparation of Z-Lys(Boc)-Val-OMe (Formula XI)

22 g of Z-Lys(Boc)-OH of Formula X, 220 ml of ethyl acetate, and 13.9 ml of N-methyl morpholine were taken into a round bottom flask and cooled to −5 to 0° C. 9.75 ml of isobutylchloroformate was added to reaction mixture dropwise at −5 to 0° C. over 30 minutes and the mixture was stirred at the same temperature for 30 minutes. 14.1 g of Val-OMe Hydrochloride was dissolved in 66 ml of dimethylformamide and cooled to 5-10° C. and neutralized with N-methyl morpholine. The neutralized H-Val-OMe solution was added dropwise to the reaction mixture at −5 to 0° C. over 30 minutes. The reaction mixture was stirred further for 1 hour at the same temperature, then the reaction temperature was brought to 25 to 35° C. and stirring continued for 5 hours. Progress of the reaction was monitored by thin layer chromatography. After completion of the reaction the reaction mass was cooled at −5 to 0° C. for 1 hour, then the reaction mass was filtered to separate the N-methyl morpholine salt. After filtration, the ethyl acetate layer was washed with 90 ml of 0.5 N hydrochloric acid solution three times. The ethyl acetate layer was then washed with 90 ml of 10% sodium bicarbonate solution three times and finally the ethyl acetate layer was washed with 100 ml of aqueous sodium chloride (brine solution) twice to neutralize the ethyl acetate layer. The ethyl acetate layer was dried with sodium sulphate and concentrated below 40° C. under vacuum to give 28.3 g of light yellow colored solid title compound.

### EXAMPLE 5

### Preparation of H-Lys(Boc)-Val-OMe (Formula XII)

28 g of Z-Lys(Boc)-Val-OMe was dissolved in 280 ml of methanol and taken into a hydrogenation flask. 3.9 ml of acetic acid was charged. 5.6 g of 10% palladium on carbon (50% water content) was added to the hydrogenation flask and a hydrogen pressure of 40-50 psi (275-345 kPa) was applied for 4 hours. Reaction completion was monitored using thin layer chromatography. After completion of the reaction, the reaction mixture was filtered through a celite bed and washed with 50 ml methanol. The filtrate was concentrated completely under vacuum below 40° C. to afford 22 g of light brown colored viscous syrup of the title compound.

### EXAMPLE 6

### Preparation of Z-(D) Trp-Lys(Boc)-Val-OMe (Formula XIV)

23.3 g of Z-(D)-Trp-OH of Formula XIII was dissolved in 233 ml ethyl acetate and 11.6 ml of N-methylmorpholine was added into it. Cooled the reaction mass to −10 to 0° C. 16.3 ml of isobutylchloroformate was added into the reaction mass dropwise at −10 to 0° C. Stirred the reaction mass for 1.5 hours at 0-5° C. 37 g of H-Lys(Boc)-Val-OMe of Formula XII was dissolved in dimethylformamide and the solution was added dropwise to the above reaction mixture at 0 to 5° C. Stirred the reaction mass at the same temperature for 1 hour, and then stirred at 25 to 35° C. for 12 hours. Confirmed the reaction completion by high performance liquid chromatography. After reaction completion the reaction mass was filtered to separate the N-methyl morpholine salt. The ethyl acetate layer was washed with 90 ml of 0.5 normal hydrochloric acid solution and again the ethyl acetate layer was washed with 90 ml 10% sodium bicarbonate solution three times and finally the ethyl acetate layer was washed with 90 ml of water to get the neutralized ethyl acetate layer. Finally the ethyl acetate layer was dried using sodium sulphate and was concentrated under vacuum at 40° C. The crude product was dissolved in 150 ml ethyl acetate and 250 ml diisopropyl ether was added slowly. The suspension was stirred for 3 hours and the obtained solid was filtered and washed with 50 ml diisopropyl ether to get 26 g of the title compound.

### EXAMPLE 7

### Preparation of H-(D) Trp-Lys(Boc)-Val-OME (Formula XV)

40 g of Z (D)-Trp-Lys(Boc)-Val-OMe of Formula XIV was dissolved 1200 ml of methanol and charged into a hydrogenation flask, and 5 ml acetic acid was added to it. 8 g of 10% palladium on carbon (50% water content) was added to the hydrogenation flask and then a hydrogen pressure of 25-30 psi (170-210 kPa) was applied for 1 hour. Confirmed the reaction completion by TLC. After reaction completion the reaction mixture was filtered through a perlite bed and washed with 50 ml methanol. The filtrate was concentrated completely under vacuum below 40° C. to give 37 g of white solid of the title compound.

### EXAMPLE 8

### Preparation of Z-Trp-OH (Formula XVII)

100 g of L-Tryptophan was dissolved in 700 ml of 1 normal sodium hydroxide solution. The reaction mixture was cooled to 0 to −5° C. 200.5 ml of 50% benzyloxycarbonyl chloride was added dropwise over 30-40 minutes at the same temperature. Another 525 ml of 1 normal sodium hydroxide solution was added dropwise at 0 to 5° C. and stirred at same temperature for 2 hours. Completion of the reaction was checked by thin layer chromatography. After the reaction was completed, the reaction mass was acidified to a pH of 4 to 5 with 5 N HCl and stirred for 1 hour at room temperature. The precipitated compound was filtered and washed with 450 ml of water three times. The compound was dried at 75° C. to 80° C. for 10 hours to get 165 g the title compound.

### EXAMPLE 9

### Preparation of Z-Trp-NH₂ (Formula XVIII)

150 g of Z-Tpr-OH was taken in 1050 ml ethyl acetate and stirred at 25 to 35° C. for 15 minutes, 99 ml of N-methyl morpholine was added, and then the reaction mass was cooled to −10 to −15° C. 69 ml of IBCF was added drop wise to the reaction mixture keeping the temperature at −10° C. After completion of IBCF addition the reaction mass was stirred for 45 minutes at −10° C. 25% aqueous ammonia solution was added to the reaction mixture dropwise over 30 minutes at −10° C. After the completion of ammonia solution addition, the reaction mixture became a clear solution. The solution was stirred at −10 to −15° C. for 3 hour. A solid precipitated during the reaction. Filtered the formed solid product under vacuum and washed with 500 ml of water. The solid was taken into 1000 ml of water, stirred at 25 to 35° C. for 45 minutes and filtered. The solid was suction dried and finally dried at 75-80° C. for 10 hours to get 105 g of the title compound.

### EXAMPLE 10

### Preparation of H-Trp-NH₂ (Formula XIX)

25 g of Z-Trp-NH₂ of Formula XVIII was dissolved in 1000 ml methanol by heating at 50-55° C. and the solution was cooled to 25 to 35° C. 5 g of 10% palladium on carbon was charged and the reaction mixture was stirred under a hydrogen atmosphere at 5-10 kg/cm² for 1 hour. Confirmed the reaction completion by high performance liquid chromatography. After reaction completion the reaction mass was filtered through a Hiflow (flux calcined diatomaceous earth) bed and the bed was washed with 50 ml methanol. Filtrate was concentrated completely below 40° C. to give 15.8 g of an off-white colored sticky mass of the title compound.

### EXAMPLE 11

### Preparation of Boc-Cys(Acm)-Trp-NH₂ (Formula XXI)

12 g of H-Trp-NH₂, 50 ml tetrahydrofuran, 18.3 g of Boc-Cys (Acm)-OH and 8.77 g of 1-hydroxybenzotriazole hydrate were taken into a round bottom flask. The suspension was stirred for 15 minutes and then cooled to 0 to 5° C. 13.3 g of 1,3-dicyclohexylcarbodiimide was added to the reaction mixture at 0 to −5° C. and stirred at the same temperature for 1 hour. The reaction temperature was brought to 25 to 35° C. for 12 hours. Confirmed the reaction completion by high performance liquid chromatography. After reaction completion the reaction mixture was cooled to 0 to 5° C. and stirred for 15 minutes. The precipitated dicyclohexylurea was filtered and filtrate concentrated completely under vacuum below 40° C. The residue was dissolved in 150 ml ethyl acetate and washed with 150 ml aqueous 0.5 N hydrochloric acid three times, followed by washing with 100 ml of water. The organic layer was washed with 90 ml of 0.5 N sodium hydroxide solution three times and 40 ml of water twice. The organic layer was dried over sodium sulphate, filtered and concentrated under vacuum below 40° C. to give 26.6 g of pale yellow colored solid of the title compound.

### EXAMPLE 12

### Preparation of Boc (D)-Phe-Cys (Acm)-Tyr-(D)Trp-Lys(Boc)-Val-OMe (Formula XXIII)

80 g of Boc (D)-Phe-Cys (Acm)-Tyr-OH of Formula VIII was dissolved in 800 ml tetrahydrofuran and 79.7 g H-(D) Trp-Lys(Boc)-Val-OMe of Formula XV was added. 19.7 g of 1-hydroxybenzotriazole hydrate was charged to the reaction mass. The reaction mixture was cooled to 0 to −5° C. 30.1 g of 1,3-dicyclohexylcarbodiimide was added at the same temperature and maintained for 1 hour. The reaction temperature was brought to ambient temperature and stirred for 5 hours. After completion of the reaction, the reaction mass was cooled to 0 to −10° C. for 1 hour. Filtered to remove the dicyclohexylurea salt and the filtrate was distilled below 40° C. under vacuum. The residue was dissolved in 800 ml of ethyl acetate and washed the organic layer with 300 ml 0.5 normal hydrochloric acid and then with 300 ml 10% sodium bicarbonate solution three times. Finally, the organic layer was washed with brine solution. Dried the organic layer with anhydrous sodium sulphate and concentrated below 40° C. under vacuum to get 137 g solid of the title compound.

### EXAMPLE 13

### Preparation of Boc (D)-Phe-Cys (Acm)-Tyr-(D) Trp-Lys(Boc)-Val-OH (Formula XXIV);

137 g of Boc (D)-Phe-Cys (Acm)-Tyr-(D) Trp-Lys(Boc)-Val-OMe was taken into 2 liters of tetrahydrofuran and stirred for 10-15 minutes. The reaction mass was cooled to 0 to 10° C. and 40.67 g of 1.5 N lithium hydroxide solution (648 ml) was added dropwise to the reaction mass. After lithium hydroxide addition the reaction mass was stirred at 25 to 35° C. for 2 hours. After completion of the reaction tetrahydrofuran was distilled from the reaction mixture under reduced pressure and the reaction mixture was diluted with 2 liters of water. The reaction mixture was washed with 500 ml diisopropyl ether twice. The aqueous layer was acidified with 15% potassium bisulphate solution to pH 3.5. Aqueous layer was extracted with 2000 ml ethyl acetate. The organic layers were combined and washed with 1500 ml water. Organic layer was separated and dried with sodium sulphate. The organic layer was filtered and concentrated under vacuum to get a sticky mass. The mass was diluted with 500 ml ethyl acetate by heating to 45° C. and then stirred at 25-35° C. for 16 hours. The obtained solid was filtered and washed with ethyl acetate (50 ml) and then solid was stirred in 100 ml of diisopropyl ether. The obtained suspension was filtered to get 59.0 g of the crude title compound. (Yield=43.7%, Purity by HPLC=73%). The crude compound was taken into 360 ml of ethyl acetate, heated to 55-60° C. for 1 hour under stirring and stirred at 25-35° C. for 2 hours. The solid was filtered and washed with diisopropyl ether to get 44 g of off-white solid of the title compound.

### EXAMPLE 14

### Preparation of Boc(D)-Phe-Cys (Acm)-Tyr-(D) Trp-Lys(Boc)-Val-Cys (Acm) Trp-NH₂ (Formula XXV)

20.5 g of Boc-Cys (Acm)-NH₂of Formula XXI was taken in 41 ml of dichloromethane and 50.1 ml of thioanisole was added. 41 ml of trifluoroacetic acid was added slowly at 25-35° C. under stirring, and stirred for 1 hour. After completion of reaction the reaction mixture was concentrated completely using high vacuum below 30 ° C. The residue was triturated with diethyl ether (500 ml) to get the white solid of 21 g TFA salt of H-Cys (Acm)-Trp-NH₂of Formula XXII .The compound was dissolved in 360 ml DMF and neutralized with 3 equivalents of N-methyl morpholine and stirred for 1 hour at 5-10° C.

36 g of Boc-(D)-Phe-Cys(Acm)-Tyr-(D)-Trp-Lys(Boc)-Val-OH was taken into 360 ml DMF and stirred for 30 minutes. The above neutralized H-Cys(Acm)-Trp-NH₂ in DMF was charged into the reaction mass. 4.8 g of 1-hydroxybenzotriazole hydrate was added and the mixture was cooled to 0 to −10° C. 6.8 g of 1-ethyl-3-(3′-dimethylaminopropyl) carbodiimidehydrochloride (EDC hydrochloride) was added to the reaction mass at 0 to −10° C. 10 ml of N-methylmorpholine was added to the reaction mixture and stirred for 24 hours at 0 to −10° C. After completion of reaction, the total reaction mass was charged into 3.5 liters of water under stirring. The precipitated white solid was filtered and leached with acetone and filtered. Dried the solid to get 38 g of the title compound.

### EXAMPLE 15

### Preparation of Vapreotide Trifluoroacetate [2TFA-(D)-Phe-Cys (Acm)-Tyr-(D) Trp-Lys-Val-Cys (Acm)-Trp-NH₂] (Formula XXVI);

30 g of Boc (D)-Phe-Cys (Acm)-Tyr-(D) Trp-Lys(Boc)-Val-Cys (Acm) Trp NH₂, was added to 90 ml of dichloromethane and stirred for 20 minutes under a nitrogen atmosphere. 23.5 ml thioanisole was added to the reaction and cooled to 0-5° C. 90 ml of trifluoroacetic acid was added to the reaction mass over 1 hour at 0-10° C. and stirred at the same temperature for 2 hours. The reaction mixture was concentrated at 25-35 ° C. to get a sticky mass. 600 ml of diethyl ether was charged into the sticky mass and stirred for 1 hour and then diethyl ether was decanted. 300 ml of diethyl ether was again charged and stirred for 1 hour. The obtained suspension was filtered and then the solid was taken into 600 ml of isopropyl alcohol and stirred at 55-60° C. for 1 hour and then stirred at 25-35° C. for 2 hours. The obtained solid was filtered and washed with 60 ml of isopropyl alcohol to obtain 24 g of an off-white title compound.

### EXAMPLE 16

### Preparation of Vapreotide Acetate

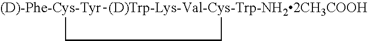

5.0 g of 2TFA-(D)-Phe-Cys (Acm)-Tyr-(D) Trp-Lys-Val-Cys (Acm)-Trp-NH₂ was added to a solution of 5 liters of 1% aqueous acetic acid and stirred to obtain dissolution at ambient temperature. 1.51 g of iodine was dissolved in 2.5 liters methanol and added slowly to the reaction mass at 25° C. over 1 hour. The reaction was continued for 4 hours and monitored by HPLC. After completion of the reaction the reaction mass was quenched with 0.05 molar sodium thiosulphate solution at 20-25° C. The solvent was concentrated until 1500 ml of methanol was distilled at 25° C. under high vacuum and then filtered to remove insoluble particles.

The filtrate was diluted with 10 liters of water and purified by preparative high performance liquid chromatography. The total volume of reaction solution was charged into Merck ODS (50 mm×250 mm) silica gel: Kromasil-RP C ^{18,}100 A, Micron size-10, Spherical silica column at 60 ml/minute for 3 hours. After loading the column, the column was stabilized by introducing 0.1% of trifluoroacetic acid at 60 ml/minute for 30 minutes. Column was eluted with 0.1% of TFA in water and acetonitrile (73:27), and then the column was washed with water and acetonitrile (50:50) for 30 minutes at 60 ml/minute. 50 ml of each fraction was collected in 30-35 fractions and all fractions are analyzed by HPLC. The column was washed with acetonitrile and water (50:50) for 30 minutes with 60 ml/minute and then washed with methanol for 25-30 minutes with 60 ml/minute. Pure fractions (97-99%) were kept aside and 350 ml volume of impure fractions (77-94%) were diluted with 1050 ml of water and then the total volume of solution was loaded into the same column at 60 ml/minute. After loading the column, the column was stabilized with 0.1% of TFA solution at 60 ml/minute for 30 minutes. Then the column was eluted with 0.1% of TFA and acetonitrile (73:27) at 60 ml/minute. After eluting the compound, the column was washed with water and acetonitrile (1:1) for 30 minutes at 60 ml/minute.

Desalting and salting done using preparative high performance liquid chromatography. The pure fractions were combined and diluted with 3 volume of milique water. The obtained total volume of solution loaded into the same column with 60 ml/minute. Column was stabilized with 5% acetic acid for 30 minutes with 60 ml/minute. After that column was washed with 0.1% of acetic acid in water and acetonitrile (70:30) with 60 ml/minute, washed with acetonitrile and water (50:50) with 60 ml/minute for 30 minutes and finally washed with methanol for 30 minutes with 60 ml/minutes. The pure fractions were combined and concentrated using a Buchi Rotavapor to remove acetonitrile. The solution was freeze-dried and isolated. 540 mg of white solid vapreoitde acetate was obtained.

### EXAMPLE 17

### Preparation of H-(D)-Trp-Lys(Boc)-Val-OH (Formula XXVIII)

4 g of H-(D)-Trp-Lys(Boc)-Val-OMe was taken in 40 ml of methanol and cooled to 0° C. 20 ml of 2N sodium hydroxide solution was added to the reaction mass slowly at the same temperature over a period of 15-20 minutes. Reaction was maintained at 0° C. for 30 minutes and at 25 to 35° C. for 2.5 hours. Confirmed the reaction completion by thin layer chromatography. After reaction completion the reaction mixture was diluted with 200 ml of water and adjusted pH to 2 with 0.5 N hydrochloric acid solution. The reaction mass was extracted into 200 ml n-butanol. The organic layer was treated with sodium sulphate and concentrated under vacuum to get 3.7 g of off-white colored title compound.

### EXAMPLE 18

### Preparation of Fmoc-(D)-Trp-Lys(Boc)-Val-OH (Formula XXIX)

3 g H-(D)-Trp-Lys(Boc)-Val-OH of Formula XXVIII was taken into 20 ml dioxane and 1.49 g sodium carbonate solution in 40 ml water was added at 25 to 35° C. Cooled the reaction mass to 0° C. 1.45 g of fluorenlymethyl chloroformate (Fmoc-Cl) dissolved in 40 ml dioxane was added to the reaction mass at 0° C. The reaction mass was maintained at 0° C. for 30 minutes and then at room temperature for 2 hours. Confirmed the reaction completion by thin layer chromatography. After reaction completion the reaction mixture was diluted with 30 ml of water. During water addition a solid was precipitated. Acidified the reaction mass with 0.5 N hydrochloric acid solution to pH 2. Aqueous layer was extracted with 60 ml ethyl acetate and the organic layer was dried with anhydrous sodium sulphate. Organic layer was concentrated under vacuum. The obtained crude mass was adsorbed on 7 g of silica. Column (3 cm diameter and 45 cm of length) was loaded with 30 g of silica gel (230-400 mesh size) and then silica-adsorbed compound was loaded into the column. Eluted the column with 1% of methanol in dichloromethane (200 ml), 3% of methanol in dichloromethane (300 ml) and 5% methanol in dichloromethane (500 ml). These fractions are collected and the solvent was distilled off to get 2 g of title compound.

### EXAMPLE 19

### Preparation of Fmoc-(D)Trp-Lys(Boc)-Val-Cys(Acm)-Trp-NH₂ (Formula XXX)

1.64 g of Boc-Cys (Acm)-Trp-NH₂ was taken in 3.28 ml of dichloromethane and 3.28 ml of trifluoroacetic acid was added slowly at 25-35° C. and stirred at 25 to 35° C. for 30 minutes. After completion of the reaction, the reaction mixture was concentrated completely below 30° C. using high vacuum and triturated with 20 ml of diethyl ether. The diethyl ether was decanted. The residue was dissolved in 10 ml dimethylformamide and neutralized with 0.873 ml of N-methyl morpholine, then stirred for 1 hour at 5-10° C. to get H-Cys (Acm)-Trp-NH₂.

2 g of Fmoc-(D)-Trp-lys(Boc)-Val-OH was taken into 20 ml tetrahydrofuran and cooled to 0° C. 0.464 g of 1-hydroxybenzotriazole hydrate was added to the reaction mass and stirred for 5 minutes at 0° C. 0.708 g of dicyclohexylcarbodiimide was added to the reaction mass at 0° C. and stirred at the same temperature for 45 minutes and then neutralized H-Cys (Acm)-Trp-NH₂ solution was added to the reaction mass at 0° C. The reaction mass was stirred at 0° C. for 30 minutes and then at room temperature for 12 hours. Confirmed the reaction completion by thin layer chromatography. After reaction completion the reaction mixture was diluted with 200 ml ethyl acetate and cooled to 0° C. and stirred for 30 minutes. Filtered the separated solid and dried to yield 1.8 g of the title compound.

### EXAMPLE 20

### Preparation of H-(D) Trp-Lys(Boc)-Val-Cys (Acm)-Trp-NH₂ (Formula XXXI)

1.9 g of Fmoc-(D) Trp-Lys(Boc)-Val-Cys(Acm)-Trp-NH₂ was taken into 10 ml acetonitrile and 10 ml of diethylamine was added dropwise at 25 to 35° C. The reaction mass was stirred at 25 to 35° C. for 5 hours. Confirmed the reaction completion by thin layer chromatography. After reaction completion the reaction mixture was concentrated under vacuum and to the crude mass was taken into 30 ml diethyl ether and stirred for 15 minutes. The diethyl ether was decanted to get 1.3 g of the title compound in solid form.

### EXAMPLE 21

### Preparation of BOC (D)-Phe-Cys(Acm)-Tyr-(D)Trp-Lys(Boc)-Val-Cys(Acm)-Trp-NH₂ (Formula XXXII)

240 mg of Boc (D)-Phe-Cys (Acm)-Tyr-OH was taken into 6 ml tetrahydrofuran. The reaction mass was cooled to 0° C. 70 mg of 1-hydroxybenzotriazole hydrate was added to the reaction mass and stirred for 5 minutes at the same temperature. 106 mg of dicyclohexylcarbodiimide was added to the reaction mass at 0° C. and stirred for 30 minutes at the same temperature.

443 mg of H-(D)Trp-Lys(Boc)-Val-Cys(Acm)-Trp-NH₂ was taken into 3 ml dimethylformamide and 0.043 ml of N-methyl morpholine was added. The solution of this pentapeptide was added to the above tripeptide reaction mass at 0° C. Stirred the reaction mass at 0° C. for 30 minutes and then at room temperature for 10 to 12 hours. Confirmed the reaction completion by thin layer chromatography. After reaction completion the reaction mixture was diluted with 50 ml of ethyl acetate. The reaction mass was filtered and filtrate was concentrated below 40° C. under vacuum to yield 220 mg of the title compound.

### EXAMPLE 22

### Preparation of Boc-(D)-Phe-Cys(Trt)-Tyr-OH

5 g of Boc-(D)-Phe-Cys (Trt)-Tyr-OMe was dissolved in 20 ml methanol, 10 ml tetrahydrofuran was added and the mixture was cooled to 0 to 10° C. 19.6 ml of aqueous 1N sodium hydroxide solution was added drop wise to the mixture over 20 minutes at 0 to 10° C. and the mixture was maintained at the same temperature for 4 hours. Progress of the reaction was monitored using thin layer chromatography. After completion of the reaction, the pH of the reaction mixture was adjusted to 3 with 25 ml of saturated aqueous potassium hydrogen sulphate solution and the mixture was extracted with 3×70 ml of ethyl acetate. The organic layer was washed with 4×50 ml of water in 4 lots and dried over sodium sulphate. The organic layer was filtered and concentrated under vacuum using a Buchi Rotavapor below 40° C. using vacuum to give 4.5 g of yellow fluffy solid.

### EXAMPLE 23

### Preparation of Boc-(D)-Phe-Cys(Trt)-Tyr-OME

12.0 gm of Boc-Cys (Trt)-Tyr-OMe was dissolved in 24 ml of dichloromethane and cooled to 0-5° C. 24 ml of trifluoroacetic acid was added slowly to the reaction mass over 15 minutes at the same temperature. The reaction mass was stirred for 40 minutes at the same temperature. Progress of the reaction was monitored by using thin layer chromatography. After completion of the reaction, the solvent was evaporated completely using high vacuum at room temperature. The residue was dissolved in 40 ml of tetrahydrofuran and cooled to 0 to 5° C. and pH was adjusted to 8 with 6.7 ml of N-methyl morpholine.

3.97 g of Boc-(D)-Phe-OH, 100 ml tetrahydrofuran, and 3.3 ml of N-methyl morpholine were mixed and cooled to 0 to 5° C. 2.9 ml of isobutyl chloroformate was added to the reaction mass dropwise over 15 minutes at the same temperature. The reaction mass was stirred further for 10 minutes and then the above neutralized H-Cys (Trt)-Tyr-OMe was added dropwise over 15 minutes at 0-5° C. and the mixture was maintained at the same temperature for 1 hour, then was brought to room temperature and maintained for 3 hours. Progress of the reaction was monitored by thin layer chromatography. After completion of the reaction, 100 ml of water and 200 ml of ethyl acetate were added to the reaction mass and stirred for 15 minutes. The organic layer was separated and washed successively with 160 ml of 0.5 N hydrochloric acid twice, 80 ml of water, 160 ml of 10% sodium bicarbonate solution twice, and finally with 320 ml of water in four lots. The organic layer was dried over sodium sulphate, filtered and solvent was concentrated completely below 40° C. to give 15 g of a yellow colored fluffy solid.

### EXAMPLE 24

### Preparation of Boc-Cys (Trt)-Tyr-OME

10 g of Boc-Cys (Trt)-OH, 80 ml of tetrahydrofuran, and 5.5 g of Tyr-OMe.hydrochloride were taken into a round bottom flask. 2.4 g of N-methyl morpholine was added to the reaction mixture at room temperature and stirred for 5 minutes. 3.2 g of 1-hydroxybenzotriazole hydrate was added to the reaction in a single lot and the reaction mixture was cooled to 0 to 5° C. 4.9 g of dicyclohexylcarbodiimide was added to the reaction at 0 to 50° C. and the mixture was stirred at the same temperature for 1 hour. The reaction temperature was brought to 25 to 35° C. and stirring continued for 14 hours. Progress of the reaction was monitored by thin layer chromatography. After completion of the reaction, the reaction mixture was cooled to −5 to −10° C. and then precipitated dicyclohexylurea was removed by filtration and the cake washed with 10 ml of cold tetrahydrofuran. The combined tetrahydrofuran was concentrated to a minimum volume using vacuum below 40° C. and the residue was dissolved in 300 ml of ethyl acetate. The solution was washed successively with 200 ml of 0.5N hydrochloric acid twice, 100 ml of water, 100 ml of 10% sodium bicarbonate solution, and finally with 300 ml of water. The organic layer was dried over sodium sulphate, filtered, and concentrated below 40° C. under vacuum to get 12.2 g of yellowish solid of the title compound.

### EXAMPLE 25

### Preparation of Boc-Cys (Trt)-Trp-NH₂

5.0 g of H-Trp-NH₂, 50 ml of tetrahydrofuran, 9 g of Boc-Cys (Trt)-OH and 6.6 g of 1-hydroxybenzotriazole hydrate were taken into a round bottom flask and the suspension was stirred for 15 minutes. The suspension was then cooled to 0 to 5° C. 7.6 g of dicyclohexylcarbodiimide was added to the mixture at 0 to 5° C. and stirred at the same temperature for 1 hour. The reaction temperature was brought to 25 to 35° C. for 12 hours. Progress of the reaction was monitored by thin layer chromatography. After completion of the reaction the reaction mass was cooled to 0 to 5° C. and stirred for 15 minutes. The precipitated dicyclohexylurea was removed by filtration and the filtrate was concentrated completely under vacuum below 40° C. The residue was dissolved in 150 ml of ethyl acetate and washed with 90 ml of aqueous 0.5 N hydrochloric acid three times, then with 30 ml of water. The solution was again washed with 90 ml of 10% sodium bicarbonate solution three times and finally with 120 ml of water in four lots. The organic layer was dried over sodium sulphate, filtered, and concentrated under vacuum below 40° C. to give a pale yellow solid. The obtained solid was dissolved in 25 ml of ethyl acetate at 50° C. and then the solution was cooled using a mixture of ice and salt to 0° C. Finally the obtained solid was filtered to get 9.0 g of Boc-Cys (Trt)-Trp-NH₂ with a purity by HPLC 97.8%.

### EXAMPLE 26

### Preparation of Boc (D)-Phe-Cys (Trt)-Tyr-(D) Trp-Lys(Boc)-Val-OME

1.5 g of Boc (D)-Phe-Cys (Trt)-Tyr-OH was dissolved in 8 ml of tetrahydrofuran and the solution was cooled to 0° C. 0.31 g of 1-hydroxybenzotriazole hydrate was added and stirred for 5 minutes, 0.475 g of dicyclohexylcarbodiimide was added at 0° C. and finally 0.95 g of H-(D)Trp-Lys(Boc)-Val-OMe was dissolved in 7 ml of tetrahydrofuran and added dropwise to the reaction mass at 0° C. The reaction mass was stirred at 0° C. for 30 minutes and then for 3 hours at 25-35° C. After completion of the reaction, the reaction mixture was cooled to 0° C. and stirred for 1 hour. Then the reaction suspension was filtered to separate the dicyclohexylurea. The filtrate was then diluted with 45 ml of ethyl acetate. The solution was washed with 30 ml of 0.1N hydrochloric acid solution twice and with 30 ml of 5% sodium bicarbonate solution twice, and finally with 30 ml of brine solution twice. The organic layer was dried with anhydrous sodium sulphate and concentrated below 40° C. under vacuum to give 2.2 g of white solid title compound.

### EXAMPLE 27

### Preparation of Boc (D)-Phe-Cys (Trt)-Tyr-(D) Trp-Lys(Boc)-Val-OH

2.1 g of Boc (D)-Phe-Cys (Trt)-Tyr-(D) Trp-Lys(Boc)-Val-OMe was taken into 21 ml of methanol and stirred for 30 minutes. The mixture was cooled to 0 to −5° C. and then 4.83 ml of 2N sodium hydroxide solution was added dropwise at the same temperature. After completion of addition, the mixture was stirred at 0° C. for 30 minutes and then at 25 to 35° C. for 4 hours. After completion of the reaction, the reaction mixture was diluted with 100 ml of water and pH adjusted with 0.5N hydrochloric acid solution to 2, then extracted with 100 ml of ethyl acetate twice. The combined organic layer was washed with 50 ml water twice and finally with 50 ml of brine solution twice. Then the organic layer was dried with sodium sulphate, filtered, and concentrated under reduced pressure below 40° C. to get 3.3 g of white solid title compound.

### EXAMPLE 28

### Preparation of Boc(D)-Phe-Cys (Trt)-Tyr-(D) Trp-Lys(Boc)-Val-Cys (Trt)-Trp-NH₂

318 mg of the hydrochloride salt of H-Cys (Trt)-Trp-NH₂ (prepared from Boc-Cys (Trt)-Trp-NH₂) was taken into 2 ml of dimethylformamide and neutralized with N-methyl morpholine to pH 8. The mixture was then stirred for 30 minutes at 5-10° C. 700 mg of Boc-(D)-Phe-Cys(Trt)-Tyr-(D)-Trp-Lys(Boc)-Val-OH was taken into 7 ml of tetrahydrofuran and stirred for 15 to 30 minutes. 135 g of EDC hydrochloride was taken into 1 ml dimethylformamide and neutralized with N-methyl morpholine to pH 8. This solution was added to the solution of Boc-(D)-Phe-Cys(Trt)-Tyr-(D)-Trp-Lys(Boc)-Val-OH. The above-neutralized H-Cys (Trt)-Trp-NH₂ in DMF was added. 96 mg of 1-hydroxybenzotriazole hydrate was added to the reaction mixture and cooled to 0° C., stirred at the same temperature for 30 minutes and then stirred at 25 to 35° C. for 5 hours. After completion of reaction the total reaction mass was diluted with 50 ml water. The reaction mass was extracted with 100 ml ethyl acetate twice. The organic layer was washed with 50 ml of 0.5N hydrochloric acid solution twice and then with 50 ml of saturated sodium bicarbonate solution twice and finally with brine solution. The organic layer was dried over sodium sulphate and concentrated below 40° C. under vacuum to get 600 mg of yellow solid title compound.

### EXAMPLE 29

### Preparation of 2TFA-(D)-Phe-Cys (Trt)-Tyr-(D) Trp-Lys-Val-Cys (Trt)-Trp-NH₂

100 mg of Boc(D)-Phe-Cys (Trt)-Tyr-(D) Trp-Lys(Boc)-Val-Cys (Trt)-Trp-NH₂ was added to 0.4 ml of dichloromethane and stirred for 5 minutes under a nitrogen atmosphere. 20 μl of thioanisole was added to the reaction mass and stirred for 5 minutes. 0.2 ml of trifluoroacetic acid was added to the reaction mass at 25 to 35° C. and stirred at the same temperature for 45 minutes. The reaction mixture was concentrated and 10 ml of diethyl ether was charged. The obtained suspension was stirred for 10 minutes and then filtered to get 75.0 mg of white solid title compound (Yield=75%, Purity by HPLC=22%), which was proceeded without purification into the next stage.

### EXAMPLE 30

### Preparation of Vapreotide Acetate

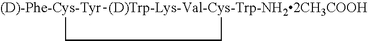 50 mg of 2TFA-(D)-Phe-Cys (Trt)-Tyr-(D) Trp-Lys-Val-Cys (Trt)-Trp-NH₂ was added to 50 ml of a solution of 1.0% aqueous acetic acid and stirred to obtain dissolution at ambient temperature. 10.3 mg of iodine was dissolved in 25 ml of methanol and this solution was added slowly to the reaction mass at 25-35° C. over 30 minutes. The reaction was stirred for 90 minutes. After completion of the reaction, the reaction mixture was quenched with 0.01M sodium thiosulphate and then concentrated to get a residue comprising 71% of the title compound by HPLC analysis.

### EXAMPLE 31

### Preparation of Boc-(D)-Phe-Cys(Bzl)-Tyr(Obzl)-OH

5.0 g of Boc-(D)-Phe-Cys (Bzl)-Tyr (OBzl)-OMe was dissolved in 30 ml of methanol and the solution was cooled to 0° C. 41 ml of aqueous 1N sodium hydroxide solution was added dropwise to the solution over 30 minutes at 0° C. and stirred for 4 hours. The reaction mixture was hazy, so 40 ml of tetrahydrofuran was added and continued maintenance at 0° C. for 4 hours. The progress of reaction was monitored by TLC. After the completion of reaction, the reaction mass was diluted with 40 ml of water. The pH of the reaction mixture was adjusted to 2 with 0.5 N hydrochloric acid and extracted with 50 ml of ethyl acetate. The organic layer was washed with 40 ml of water twice and dried over sodium sulphate. The organic layer was filtered and concentrated at 35° C. under vacuum to give 2.6 g of fluffy solid title compound.

### EXAMPLE 32

### Preparation of Boc-(D)-Phe-Cys(Bzl)-Tyr(Obzl)-OMe

1 g of Boc-Cys-(Bzl)-Tyr (OBzl)-OMe was dissolved in 4 ml of dichloromethane and 4 ml of trifluoroacetic acid was added slowly to the reaction mixture over 15 minutes at 25 to 35° C. The reaction mixture was stirred for 1 hour at the same temperature. Progress of the reaction was monitored by thin layer chromatography. After completion of the reaction, the solvent was evaporated completely using high vacuum at 25 to 35 ° C. The residue was dissolved in 5 ml of tetrahydrofuran cooled to 0 to 5° C. and pH was adjusted to 8 with N-methyl morpholine to get 34.7 g of neutralized H-Cys(Bzl)-Tyr(OBzl)-OMe.

0.37 g Boc (D) Phe-OH, 5 ml of tetrahydrofuran, and 0.78 ml of N-methyl morpholine were mixed and cooled to 0 to 5° C. 0.27 ml of isobutyl chloroformate was added to the reaction mass dropwise over 15 minutes at the same temperature. The reaction mass was stirred further for 10 minutes and then the above H-Cys (Bzl)-Tyr (OBzl)-OMe solution was added dropwise over 15 minutes at 0-5° C. and maintained at that temperature for another hour. The reaction temperature was brought to 25 to 35° C. and continued maintenance for 12 hours. Progress of the reaction was monitored by thin layer chromatography. After completion of the reaction, the reaction mass was cooled to 0° C. and solids were removed by filtration. The filtrate was evaporated under vacuum. The residue was dissolved in 50 ml of ethyl acetate and washed successively with 20 ml of 0.5 N hydrochloric acid twice, 20 ml of water twice, 20 ml of 10% sodium bicarbonate solution twice, and finally with 20 ml of water. The organic layer was dried over sodium sulphate, filtered and solvent was concentrated completely at 40° C. to give 1.2 g of fluffy solid title compound.

### EXAMPLE 33

### Preparation of Boc-Cys (Bzl)-Tyr(OBzl)-OMe

2.0 g of Boc-Cys (Bzl)-OH and 20 ml of tetrahydrofuran were taken into a round bottom flask. Separately, 2.26 g of H-Tyr (OBzl)-OMe. hydrochloride and 10 ml of tetrahydrofuran were charged into another round bottom flask and 1 ml of N-methyl morpholine was added at 10° C. and stirred for 15 minutes, then this was added to the Boc-Cys (Bzl)-OH mixture at 0-10° C. 1.45 g of dicyclohexylcarbodiimide was added at 0 to 5° C. and stirred at the same temperature for 1 hour. The progress of the reaction was monitored using thin layer chromatography. The temperature of the reaction mixture was brought to 25 to 35° C. and reaction continued for 14 hours. After completion of the reaction, the reaction mass was cooled to −5 to −10° C. and stirred for 1 hour. Dicyclohexylurea was removed by filtration and the cake was washed with 10 ml of cold tetrahydrofuran. The tetrahydrofuran filtrate was concentrated at below 40° C. to a volume of 20 ml using vacuum below 40° C. and the residue was dissolved in 50 ml of ethyl acetate. The solution was washed successively with 20 ml of 0.5N hydrochloric acid twice, 10 ml water, 20 ml of 10% sodium bicarbonate solution twice, and finally with 30 ml of water three times. The organic layer was dried over sodium sulphate, filtered and concentrated at below 40° C. to get 3.8 g of the title compound.

### EXAMPLE 34

### Preparation of Boc-Cys(Bzl)-Trp-NH₂

1.56 g of H-Trp-NH₂, 20 ml tetrahydrofuran, 2.0 g of Boc-Cys (Bzl)-OH and 1.29 g of 1-hydroxybenzotriazole hydrate were taken into a round bottom flask. The mixture was stirred for 15 minutes and then cooled to 0 to −5° C. 1.58 g of dicyclohexylcarbodiimide was added to the reaction mixture at 0 to −5° C. and stirred at the same temperature for 1 hour. The reaction temperature was brought to 25 to 35° C. and stirred for 12 hours. Progress of the reaction was monitored using thin layer chromatography. After completion of the reaction, the reaction mixture was cooled to 0 to 5° C. and stirred for 15 minutes. The precipitated dicyclohexylurea was removed by filtration and the filtrate solvent evaporated completely under vacuum below 40° C. The residue was dissolved in 50 ml of ethyl acetate and washed with 30 ml of aqueous 0.5 N hydrochloric acid, and finally with 40 ml of water twice. The organic layer was dried over sodium sulphate, filtered and the solvent was concentrated under vacuum below 40° C. to give a pale yellow solid. The crude solid was dissolved in 40 ml of ethyl acetate at 50° C. and cooled to 0° C. The obtained suspension was stirred for 3 hours and finally the solid was filtered. The resultant solid was dried at 55° C. for 4 hours under vacuum to get 1.2 g of the title compound.

### EXAMPLE 35

### Preparation of Boc (D)-Phe-Cys (Bzl)-Tyr-(D) Trp-Lys(Boc)-Val-OMe

2.5 g of Boc (D)-Phe-Cys(Bzl)-Tyr-OH was dissolved in 10 ml of tetrahydrofuran and the solution was cooled to 0° C. 0.57 g of 1-hydroxybenzotriazole hydrate was added and stirred for 5 minutes, then 0.86 g of dicyclohexylcarbodiimide was added at 0° C. and stirred at 0° C. for 30 minutes. 1.9 g of H-(D)Trp-Lys(Boc)-Val-OMe was dissolved in 15 ml tetrahydrofuran and added dropwise to the reaction mass at 0° C. The reaction mass was stirred at 0° C. for 30 minutes and then for 12 hours at 25-35° C. After completion of the reaction, the reaction mass was cooled to 0° C. and stirred for 1 hour. The reaction mass was filtered to separate dicyclohexylurea. The filtrate was diluted with 50 ml ethyl acetate. The filtrate was washed with 30 ml of 0.1N hydrochloric acid solution twice, with 30 ml of 10% sodium bicarbonate solution twice, and finally with 30 ml of brine solution twice. The organic layer was dried with anhydrous sodium sulphate and concentrated below 40° C. under vacuum to get 3.7 g of the title compound.

### EXAMPLE 36

### Preparation of Boc (D)-Phe-Cys (Bzl)-Tyr-(D) Trp-Lys(Boc)-Val-OH

3.6 g of Boc (D)-Phe-Cys (Bzl)-Tyr-(D) Trp-Lys(Boc)-Val-OMe was taken into a round bottom flask and 36 ml of methanol was added. The reaction mass was stirred for 30 minutes, and then cooled to 0 to 5° C. 8.75 ml of 2N sodium hydroxide solution was added dropwise to the reaction mass at the same temperature. After completion of addition, the reaction mass was stirred at 0-5° C. for 15 minutes and then at 25 to 35° C. for 2 hours. After completion of the reaction, the reaction mass was diluted with 50 ml water and acidified with 50 ml of 0.5 N hydrochloric acid to pH 2. The reaction mass was extracted with 100 ml ethyl acetate. The organic layer was washed with 50 ml water twice and finally with 50 ml of brine solution twice. The organic layer was then dried with sodium sulphate. The organic layer was filtered and then distilled under reduced pressure below 40° C. to give 3.2 g of the crude title compound.

### EXAMPLE 37

### Preparation of Boc (D)-Phe-Cys (Bzl)-Tyr-(D) Trp-Lys(Boc)-Val-Cys (Bzl)-Trp-NH₂

437 mg of Boc-Cys(Bzl)-Trp-NH₂ was taken into 1.8 ml of dichloromethane. 0.874 ml of triflouroacetic acid was added dropwise to the reaction mass at 25-35° C. The reaction mass was stirred for 30 minutes at 25 to 35° C. The reaction mass was concentrated under vacuum to get a sticky mass, and the mass was triturated with diethyl ether to get solid product. The solid was taken into 2 ml of dimethylformamide and neutralized with N-methyl morpholine until the pH of the reaction mass was 8. The reaction mass was stirred for 30 minutes at 5-10° C.

900 mg of Boc-(D)-Phe-Cys(Bzl)-Tyr-(D)-Trp-Lys(Boc)-Val-OH was taken in 5 ml of tetrahydrofuran and stirred for 20 minutes. The mixture was cooled to −5 to 0° C. 120 mg of 1-hydroxybenzotriazole hydrate was added. 168 mg of EDC: hydrochloric acid was taken into 1 ml of dimethylformamide and neutralized with N-methyl morpholine to pH 7.8 and added to the mixture. The mixture was stirred for 30 minutes at −5 to 0° C.

The neutralized H-Cys(Bzl)-Trp-NH2 in dimethylformamide (prepared above) was added to the mixture. The mixture was stirred at −5 to 0° C. for 30 minutes and then at 25 to 35° C. for 10 hours. After the completion of the reaction, the reaction mass was concentrated under vacuum and then diluted with 50 ml of ethyl acetate. The ethyl acetate layer was washed with 40 ml of 0.5N hydrochloric acid solution twice, 40 ml of 10% sodium bicarbonate solution twice, and finally with 40 ml of brine solution twice. The organic layer was dried over sodium sulphate. The organic layer was filtered and solvent was evaporated under vacuum below 40° C. to give 750 mg of the title compound.

### EXAMPLE 38

### Preparation of TFA Salt 2TFA-(D)-Phe-Cys (Bzl)-Tyr-(D)Trp-Lys-Val-Cys (Bzl)-Trp-NH₂

250 mg of Boc-(D)-Phe-Cys (Bzl)-Tyr-(D)Trp-Lys(Boc)-Val-Cys (Bzl)-Trp-NH₂ was added to 1 ml of dichloromethane and stirred for 15 to 20 minutes under a nitrogen atmosphere. 58 mg of thioanisole was added. 0.5 ml of trifluoroacetic acid was added at 25 to 35° C. and stirred at the same temperature for 45 minutes. The reaction mixture was concentrated below 40° C. followed by charging 5 ml of diethyl ether and stirring for 10 minutes, and then the obtained off-white solid was filtered to get 250 mg of the title compound.

### EXAMPLE 39

### Preparation of Vapreotide Acetate (Benzyl Route)

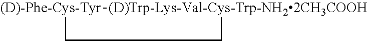

Small metallic sodium pieces were added to 100 ml of distilled liquid ammonia at −70° C. until a blue color developed. The mixture was stirred for 5 minutes. 50 mg of 2TFA-(D)-Phe-Cys (Bzl)-Tyr-(D) Trp-Lys-Val-Cys (Bzl)-Trp-NH₂ was dissolved in 1 ml of dimethylformamide and this solution was added to the Na/liquid NH₃ solution, and the blue color disappeared. The reaction mass was stirred for 5 minutes and then 2 drops of acetic acid was added to reaction mass and stirred for 5 minutes. 20 mg of potassium ferrocyanide was added to reaction mass. The reaction mass was stirred for 60 minutes at 25-35° C. Liquid ammonia was totally evaporated as the reaction mass came to 25-35° C. pH of the reaction mass was adjusted to 5 using 0.5 N hydrochloric acid solution. The obtained solution was purified by using preparative column chromatography as mentioned in example 16 to get 1 mg of title compound.

1. A solution phase process for preparing vapreotide or a salt thereof, comprising:
a) reacting Boc-(D)-Phe-Cys(PG)-TyrOH, where Boc is tert-butylcarbonate and PG is a protecting group, with H-(D)-Trp-Lys(Boc)-Val-OMe to form Boc-(D)-Phe-Cys(PG)-Tyr-(D)-Trp-Lys(Boc)-Val-OMe;
b) reacting Boc-(D)-Phe-Cys(PG)-Tyr-(D)-Trp-Lys(Boc)-Val-OMe with a base to form Boc-(D)-Phe-Cys(PG)-Tyr-(D)-Trp-Lys(Boc)-Val-OH;
c) reacting Boc-(D)-Phe-Cys(PG)-Tyr-(D)-Trp-Lys(Boc)-Val-OH with H-Cys(PG)-Trp-NH₂ to form Boc-(D)-Phe-Cys(PG)-Tyr-(D)-Trp-Lys(Boc)-Val-Cys(PG)-Trp-NH₂;
d) reacting Boc-(D)-Phe-Cys(PG)-Tyr-(D)-Trp-Lys(Boc)-Val-Cys(PG)-Trp-NH₂ with an acid to form NH₂-(D)Phe-Cys(PG)-Tyr-(D)-Trp-Lys-Val-Cys(PG)-Trp-NH₂; and
e) reacting NH₂-(D)Phe-Cys(PG)-Tyr-(D)-Trp-Lys-Val-Cys(PG)-Trp-NH₂ with iodine in an acidic environment to form vapreotide.

2. The process of claim 1, wherein a solution comprising Boc-(D)-Phe-Cys(PG)-Tyr-(D)-Trp-Lys(Boc)-Val-OMe obtained in a) is progressed directly into b).

3. The process of claim 1, wherein a solution comprising Boc-(D)-Phe-Cys(PG)-Tyr-(D)-Trp-Lys(BOC)-Val-OH obtained in b) is progressed directly into c).

4. The process of claim 1, wherein one or both of reactions in a) and c) occur in the presence of a carbodiimide compound coupling reagent.

5. The process of claim 1, wherein Boc-(D)-Phe-Cys(PG)-TyrOH is prepared by a process comprising
a) reacting Boc-Cys(PG)OH with H-Tyr-OMe.HCl to form Boc-Cys(PG)-Tyr-OMe;
b) reacting Boc-Cys(PG)-Tyr-OMe with H-Cys(PG)-Tyr-OMe to form Boc-(D)-Phe-Cys(PG)-Tyr-OMe; and
c) reacting Boc-(D)-Phe-Cys(PG)-Tyr-OMe with an acid.

6. The process of claim 5, wherein a solution comprising Boc-Cys(PG)-Tyr-OMe from a) is progressed directly into b).

7. The process of claim 5, wherein a solution comprising Boc-(D)-Phe-Cys(PG)-Tyr-OMe from b) is progressed directly into c).

8. The process of claim 1, wherein H-(D)-Trp-Lys(Boc)-Val-OMe is prepared by a process comprising:
a) reacting Val-OMe with Z-Lys(Boc)-OH, where Z is a benzyloxycarbonyl group, to form Z-Lys(Boc)-Val-OMe;
b) treating Z-Lys(Boc)-Val-OMe with an acid and a deprotecting reagent in the presence of hydrogen to form H-Lys(Boc)-Val-OMe;
c) reacting H-Lys(Boc)-Val-OMe with Z-(D)-Trp-OH to form Z-(D)-Trp-Lys(Boc)-Val-OMe; and
d) reacting Z-(D)-Trp-Lys(Boc)-Val-OMe with an acid and deprotecing reagent in the presence of hydrogen.

9. The process of claim 8, wherein a solution comprising Z-Lys(Boc)-Val-OMe from a) is progressed directly into b).

10. The process of claim 8, wherein a solution comprising Z-(D)-Trp-Lys(Boc)-Val-OMe from c) is progressed directly into d).

11. The process of claim 8, wherein a solution comprising H-(D)-Trp-Lys(Boc)-Val-OMe from d) is progressed directly into a reaction to form vapreotide.

12. The process of claim 8, wherein one or both of reactions in a) and c) occur in the presence of a carbodiimide compound coupling reagent.

13. The process of claim 1, wherein H-Cys(PG)-Trp-NH₂ is prepared by a process comprising:
a) reacting H-Trp-OH with benzyloxycarbonyl chloride to form Z-Trp-OH, where Z is a benzyloxycarbonyl group;
b) reacting Z-Trp-OH with an alkyl chloroformate; then with ammonia to form Z-Trp-NH₂;
c) reducing Z-Trp-NH₂ to form H-Trp-NH₂;
d) reacting H-Trp-NH₂ with Boc-Cys(PG)-Trp-NH₂ to form Boc-Cys(PG)-Trp-NH₂; and
e) reacting Boc-Cys(PG)-Trp-NH₂ with an acid.

14. A solution phase process for preparing vapreotide or a salt thereof, comprising:
a) reacting H-(D)-Trp-Lys(Boc)-Val-OMe, where Boc is tert-butylcarbonate, with a base to form H-(D)-Trp-Lys(Boc)-Val-OH;
b) reacting H-(D)-Trp-Lys(Boc)-Val-OH with fluorenlymethyl chloroformate to form Fmoc-(D)-Trp-Lys(Boc)-Val-OH, where Fmoc is a fluorenlymethoxycarbonyl group;
c) reacting Fmoc-(D)-Trp-Lys(Boc)-Val-OH with TFA-Cys(Acm)-Trp-NH2, where TFA is trifluoroacetic acid and Acm is N-(hydroxymethyl)acetamide, to form Fmoc-(D)-Trp-Lys(Boc)-Val-Cys (Acm)-Trp-NH₂;
d) reacting Fmoc-(D)-Trp-Lys(Boc)-Val-Cys (Acm)-Trp-NH₂ with a base to form H-(D)-Trp-Lys(Boc)-Val-Cys(Acm)-Trp-NH₂;
e) coupling H-(D)-Trp-Lys(Boc)-Val-Cys(Acm)-Trp-NH₂ with Boc-(D)-Phe-Cys(Acm)-Tyr-OH to form Boc-(D)-Phe-Cys(Acm)-Tyr-(D)-Trp-Lys(Boc)-Val-Cys(Acm)-Trp-NH₂;
f) reacting Boc-(D)-Phe-Cys(Acm)-Tyr-(D)-Trp-Lys(Boc)-Val-Cys(Acm)-Trp-NH₂ with an acid to form NH₂-(D)Phe-Cys(Acm)-Tyr-(D)-Trp-Lys-Val-Cys(Acm)-Trp-NH₂; and
g) reacting NH₂-(D)Phe-Cys(Acm)-Tyr-(D)-Trp-Lys-Val-Cys(Acm)-Trp-NH₂ with iodine in the presence of an acid to form vapreotide.

15. The process of claim 14, wherein a solution comprising H-(D)Trp-Lys(Boc)-Val-OH from a) is progressed directly into b).

16. The process of claim 14, wherein a solution comprising H-(D)-Trp-Lys(Boc)-Val-Cys(Acm)-Trp-NH₂ from d) is progressed directly into e).

17. The process of claim 14, wherein one or both of reactions in c) and e) occur in the presence of a carbodiimide compound coupling reagent.

18. A compound having the formula H-Lys(Boc)-Val-OMe, where Boc is tert-butylcarbonate.

19. A compound having the formula Z-(D)-Trp-Lys(Boc)-Val-OMe, where Z is a benzyloxycarbonyl group and Boc is tert-butylcarbonate.

20. A compound having the formula Boc-(D)-Phe-Cys(Acm)-Tyr-OH, where Acm is N-(hydroxymethyl)acetamide and Boc is tert-butylcarbonate.

21. A compound having the formula Boc-(D)-Phe-Cys(Acm)-Tyr-(D)-Trp-Lys(Boc)-Val-Cys(Acm)-Trp-NH₂, where Acm is N-(hydroxymethyl)acetamide and Boc is tert-butylcarbonate.
